(19) United States
(12) Patent Application Publication
(10) Pub. No.: US 2015/0088043 A1
(43) Pub. Date: Mar. 26, 2015

(54) ACTIVELY CONTROLLED WEARABLE ORTHOTIC DEVICES AND ACTIVE MODULAR ELASTOMER SLEEVE FOR WEARABLE ORTHOTIC DEVICES

(71) Applicants: Eugene C. Goldfield, Sherborn, MA (US); Yong-lae Park, Pittsburgh, PA (US); Bor-rong Chen, Medford, MA (US); Carmel Majidi, Pittsburgh, PA (US); Robert J. Wood, Cambridge, MA (US); Radhika Nagpal, Cambridge, MA (US)

(72) Inventors: Eugene C. Goldfield, Sherborn, MA (US); Yong-lae Park, Pittsburgh, PA (US); Bor-rong Chen, Medford, MA (US); Carmel Majidi, Pittsburgh, PA (US); Robert J. Wood, Cambridge, MA (US); Radhika Nagpal, Cambridge, MA (US)

(73) Assignee: PRESIDENT AND FELLOWS OF HARVARD COLLEGE, Cambridge, MA (US)

(21) Appl. No.: 14/342,254

(22) PCT Filed: Sep. 1, 2012
(86) PCT No.: PCT/US2012/053569

§ 371 Date: Jun. 24, 2014

Related U.S. Application Data
(60) Provisional application No. 61/529,961, filed on Sep. 1, 2011.

(51) Int. Cl. A61F 5/01

(52) U.S. Cl. CPC A61F 5/01; A61F 2210/008; A61F 2210/0085; A61F 2210/0076
USPC 602/6

(57) ABSTRACT

A flexible orthotic device includes two or more active components embedded in a sheet material. Each active component can include a controller and one or more actuation elements controlled by the controller. The two or more active components can communicate with each other and cause the active components to contract and dynamically change the structural characteristics of the orthotic device. By coordinating the motion of two or more active components, the flexible orthotic device can be programmed to assist or resist the motion of a subject wearing the device. The orthotic device can be effectively employed to provide locomotion assistance, gait rehabilitation, and gait training. Similarly, the orthotic device may be applied to the wrist, elbow, torso, or any other body part. The active components may be actuated to effectively transmit force to a body part, such as a limb, to assist with movement when desired. Additionally or alternatively, the active components may also be actuated to provide support of varying rigidity for the corresponding body part. The active components can be actuated to provide specialized learning tasks to enhance exploratory learning.

### CROSS REFERENCE TO RELATED APPLICATIONS

This application is related to U.S. Provisional Patent Application No. 61/225,788, filed Jul. 15, 2009, and PCT Application PCT/US PCT/US10/42106, filed Jul. 15, 2010, the contents of which are incorporated by reference, herein, in their entirety. This application claims the benefit of, and priority to, U.S. Provisional Patent Application No. 61/529,961, filed on Sep. 1, 2011, entitled “ACTIVELY CONTROLLED WEARABLE ORTHOTIC DEVICES,” the entire disclosure of which is hereby incorporated herein by reference.

This invention was made with U.S. government support under CNS 0932015 awarded by the National Science Foundation. The U.S. government has certain rights in the invention.

### BACKGROUND OF THE INVENTION

1. Field of the Invention

The present invention generally relates to orthotic devices, and, more particularly, to an actively controlled, wearable orthotic device having an array of modular components that can dynamically change the structural characteristics of the orthotic device and assist with changing the orientation and locomotion of the corresponding body part of a subject. The individual modular components can work in concert by communicating with each other to provide a broad range of simple and complex motion.

Also, the present invention relates to an adaptive soft orthotic device that performs motion sensing and production of assistive forces with a modular, pneumatically-driven, hyper-elastic composite. The device may include a material that can be wrapped around a joint to allow simultaneous motion sensing and active force response through shape and rigidity control. A monolithic elastomer sheet can contain a series of miniaturized pneumatically-powered McKibben-type actuators that exert tension and enable adaptive rigidity control. The elastomer can be embedded with conductive liquid channels that detect strain and bending deformations induced by the pneumatic actuators. In addition, the proposed system can be modular and can be configured for a diverse range of motor tasks, joints, and human subjects. This modular functionality can be accomplished with a decentralized network of self-configuring nodes that manage the collection of sensory data and the delivery of actuator feedback commands. The present disclosure describes the design of the soft orthotic device as well as actuator and sensor components. The characterization of the individual sensors, actuators, and the integrated device is also presented.

Further, the present invention relates to a combination of a sensor (such as a hyper elastic strain sensor) with an actuator (such as a miniature pneumatic actuator) as a single functional unit that can be organized into different combinations to provide a modular multifunction system. To achieve the modular multifunction system, a communication system is provided, which can be adapted to receive information from the sensor and/or the actuator, to analyze such information, to recognize patterns, and to send signals to the system so that the system operates in a manner to form desired shapes of the device, where the desired shapes can correspond with, replace or enhance a particular human motion.

2. Description of Related Art

Typically, developing infants produce well organized general movements. These “general” movements involve the whole body in a variable sequence of arm, leg, trunk, and neck movements. They wax and wane in intensity, force, and speed. Rotations along the axis of the limbs and slight changes in movement direction make them complex, fluent, and elegant (Einspieler & Prechtl, 2005). An absence of fluid general movements (what Prechtl calls “fidgety”) in the perinatal period is predictive of cerebral palsy in childhood. Currently, there is no way to “teach” developing infants with brain injuries what they cannot learn on their own.

Further, conventional treatments of gait pathologies, such as drop-foot, spasticity, contractures, ankle equinus, crouch gait, etc., associated with neuromuscular disorders, such as cerebral palsy, may employ a passive mechanical brace to support the body parts involved in balance and gait. Depending on the severity of the gait pathology, the brace may be applied to the hip, knee, ankle, or any combination thereof to improve balance and gait and to help prevent injuries.

While passive mechanical braces may provide certain benefits, they may also lead to additional medical problems. For example, a typical treatment for preventing the foot from dragging on the ground in the case of drop-foot requires the patient to use an ankle foot orthotic (AFO). Rigid versions of the AFO constrain the ankle to a specific position, while hinged or flexible versions of the AFO allow limited plantar and dorsal flexion. By limiting the range of ankle motion, the toe can clear the ground thus allowing gait to progress more naturally and promoting increased walking speeds, increased step lengths, and reduced energy consumption during gait when compared to a subject without the device. However, the use of the AFO may result in a reduction in power generation at the ankle, as the AFO limits active plantar flexion. Additionally, the use of the AFO may lead to increased transverse-plane rotation on the knee depending on the AFO alignment. As such, the use of the AFO may yield new gait abnormalities and knee problems over time. Moreover, rigid versions of the AFO may lead to disuse atrophy of the muscles, such as the tibialis anterior muscle, potentially leading to long-term dependence on the AFO.

To address the problems caused by the rigidity of conventional orthotic devices, attempts have been made to increase the flexibility of orthotic devices and to allow a greater range of motion. However, some designs for flexible orthotic devices often fail to provide sufficient flexibility to overcome the disadvantages of a typical rigid device and to provide a desired range of motion. Moreover, although other designs of orthotic devices may provide sufficient flexibility, they generally fail to take into account the individual characteristics of the subject's movement and the subject's other possible pathological conditions. Indeed, designs for flexible orthotic devices are typically passive. As such, the devices cannot be dynamically adjusted to accommodate characteristics specific to a subject during the subject's movement. In addition, the devices cannot be dynamically adjusted to accommodate the changing needs of the subject over a period of use. In general, typical flexible orthotic devices fail to provide appropriate levels of support and assistance during the subject's movement.

Also, an estimate of 11.4% of Americans 15 years or older struggle with walking or climbing stairs and about 8% have limited upper body mobility (E. Steinmetz, Americans with disabilities: 2002. Washington D.C.: U.S. Census Bureau, 2006). These physical disabilities may be congenital or acquired and arise from a broad range of neuromuscular and skeletal impairments. Common sources include stroke, traumatic brain injury, spinal injury, amputation, muscular dystrophy (MD), and cerebral palsy (CP). In many cases, motor impairment can dramatically impact health, livelihood, and quality of life and lead to secondary medical disorders and physical dependency (F. Miller, Cerebral Palsy, 1st ed. Springer, January 2005).

Assistive technologies such as electrically powered prosthetics (S. K. Au, J. Weber, and H. Herr, “Powered ankle-foot prosthesis improves walking metabolic economy,” IEEE Trans. Rob., vol. 25, no. 1, pp. 51-66, February 2009; M. F. Eilenberg, H. Geyer, and H. Herr, “Control of a powered ankle-foot prosthesis based on a neuromuscular model,” IEEE Trans. Neural Syst. Rehabil. Eng., vol. 18, no. 2, pp. 164-173, April 2010; B. G. Nascimento, C. B. S. Vimieiro, D. A. P. Nagem, and M. Pinotti, “Hip orthosis powered by pneumatic artificial muscle: Voluntary activation in absence of myoelectrical signal,” Artif. Organs, vol. 32, no. 3, pp. 317-322, 2008), orthotic exoskeletons (H. M. Herr and R. D. Kornbluh, “New horizons for orthotic and prosthetic technology: artificial muscle for ambulation,” in Proc. SPIE, vol. 5385, 2004, pp. 1-9; D. P. Ferris, J. M. Czerniecki, and B. Hannaford, “An ankle-foot orthosis powered by artificial peumatic muscles,” J. Appl. Biomech., vol. 21, pp. 189-197, 2005; Y.-L. Park, B. Chen, D. Young, L. Stirling, R. J. Wood, E. Goldfield, and R. Nagpal, “Bio-inspired active soft orthotic device for ankle foot pathologies,” in Proc. IEEE/RSJ Int. Conf Intell. Rob. Syst., San Francisco, Calif., September 2011; L. Stirling, C. Yu, J. Miller, R. J. Wood, E. Goldfield, and R. Nagpal, “Applicability of shape memory alloy wire for an active, soft orthotic,” J. Mater. Eng. Perform., vol. 20, no. 4-5, pp. 658-662, 2011), and medical robotics can dramatically improve motor independence and rehabilitation. Such technologies have the potential to restore or compensate for lost function and, in the case of congenital impairments like MD or CP, promote normal motor development. Progress, however, will depend on new assistive technologies that are comfortable to wear, safe, and compatible with human motion and mechanics. For most applications, this requires a transition from conventional electronics and motors, which are rigid and inextensible, to active materials that are elastically stretchable and match the soft compliance of human skin and tissue.

Known active orthotics systems are described in H. M. Herr and R. D. Kornbluh, “New horizons for orthotic and prosthetic technology: artificial muscle for ambulation,” in Proc. SPIE, vol. 5385, 2004, pp. 1-9; Y.-L. Park, B. Chen, D. Young, L. Stirling, R. J. Wood, E. Goldfield, and R. Nagpal, “Bio-inspired active soft orthotic device for ankle foot pathologies,” in Proc. IEEE/RSJ Int. Conf Intell. Rob. Syst., San Francisco, Calif., September 2011; L. Stirling, C. Yu, J. Miller, R. J. Wood, E. Goldfield, and R. Nagpal, “Applicability of shape memory alloy wire for an active, soft orthotic,” J. Mater. Eng. Perform., vol. 20, no. 4-5, pp. 658-662, 2011; H. Krebs, N. Hogan, W. Durfee, and H. Herr, “Rehabilitation robotics, orthotics, and prosthetics; chapter 48,” Textbook of Neural Repair and Rehabilitation (M. E. Selzer, S. Clarke, L. G. Cohen, P. W. Duncan, and F. H. Gage), Cambridge University Press, 2005.

Known exoskeleton systems are described in D. P. Ferris, J. M. Czerniecki, and B. Hannaford, “An ankle-foot orthosis powered by artificial peumatic muscles,” J. Appl. Biomech., vol. 21, pp. 189-197, 2005; K. E. Gordon, G. S. Sawicki, and D. P. Ferris, “Mechanical performance of artificial pneumatic muscles to power an ankle-foot orthosis,” J. Biomech., vol. 39, pp. 1832-1841, 2006. These known systems are typically rigid and non-modular and have a single function.

Known soft robotics systems are described in Y.-L. Park, B. Chen, D. Young, L. Stirling, R. J. Wood, E. Goldfield, and R. Nagpal, “Bio-inspired active soft orthotic device for ankle foot pathologies,” in Proc. IEEE/RSJ Int. Conf. Intell. Rob. Syst., San Francisco, Calif., September 2011; C. Majidi, R. Kramer, and R. J. Wood, “A non-differential elastomer curvature sensor for softer-than-skin electronics,” Smart Mater. Struct., vol. 20, no. 10, p. 105017, 2011; K. Noda, E. Iwase, K. Matsumoto, and I. Shimoyama, “Stretchable liquid tactile sensor for robot joints,” in Proc. IEEE Int. Conf. Rob. Autom., Anchorage, Ak., May 2010; Y.-L. Park, C. Majidi, R. Kramer, P. Berard, and R. J. Wood, “Hyperelastic pressure sensing with a liquid-embedded elastomer,” J. Micromech. Microeng., vol. 20, no. 12, 2010; Y.-L. Park, B. Chen, and R. J. Wood, “Design and fabrication of soft artificial skin with using embedded microchannels and liquid conductors,” IEEE Sens. J., vol. 12, no. 8, pp. 2711-2718, 2012.

Known embedded sensing systems are described in V. Giurgiutiu and A. N. Zagrai, “Embedded self-sensing piezoelectric wafer active sensors for structural health monitoring,” Trans. ASME, J. Vibr. Acoust., vol. 124, no. 1, pp. 116-125, 2002; A. Kadowaki, “Development of soft sensor exterior embedded with multi-axis deformable tactile sensor system,” in Proc. IEEE Int. Symp. Rob. Hum. Interact. Commun., Toyama, Japan, September 2009, pp. 1093-1098; Y.-L. Park, S. C. Ryu, R. J. Black, K. Chau, B. Moslehi, and M. R. Cutkosky, “Exoskeletal force-sensing end-effectors with embedded optical fiber-bragg-grating sensors,” IEEE Trans. Rob., vol. 25, no. 6, pp. 1319-1331, December 2009; Y.-L. Park, S. Elayaperumal, B. Daniel, S. C. Ryu, M. Shin, J. Savall, R. J. Black, B. Moslehi, and M. R. Cutkosky, “Real-time estimation of 3-d needle shape and deflection for mri-guided interventions,” IEEE/ASME Trans. Mechatron., vol. 15, no. 6, pp. 906-915, December 2010.

### SUMMARY OF THE INVENTION

It may be possible to restore function following perinatal brain injury by providing infants with enriched opportunities for guided exploratory learning. Exploratory learning during spontaneous kicking gives infants an opportunity for learning about coordination, dynamic stability, and harnessing available potential energy. During supine kicking, infants learn the different ways that the hip, knee, and ankle joint rotations covary in order to maintain a whole leg posture (e.g., hip toe length). Brain-injured infants may have difficulty producing a large covariation set of joint angles from which to extract a solution to the dynamics. During supine kicking, infants learn the boundaries of body stability in a gravitational field. Brain-injured infants may have overly stiff muscles that interfere with learning about body stability, and may have difficulty mapping sensory information onto body motion. During supine kicking, infants learn that the limbs are compliant pendula, and that by activating the muscles at the peak of available potential energy, it takes less muscular effort to initiate and sustain motion. Brain-injured infants may have limited available potential energy, and may have difficulty in using sensory information about body motion.

What infants learn before they walk is that (a) it is possible to produce different combinations of hip, knee, and ankle joint rotations that maintain the whole leg in a stable posture (e.g., relatively constant length between hip and ankle joints); (b) the body is influenced by gravitational and other forces; (c) by using the muscles in cooperation with the forces acting on the body, it is possible to use less muscular effort to move body parts.

In accordance with various embodiments of the present invention, a cyberphysical system (CPS) integrates computation and physical processes (Lee & Seshia, 2011) to provide guided learning opportunities for children with brain injuries. A second skin orthotic device according to the present invention can provide sensor-guided assistive actuation during rehabilitation. For rehabilitation, second skin orthotic device can be used with a family of developmentally appropriate special-purpose mechanical systems (scaffolds) that provide specific learning experiences and therapies for a child wearing second skin orthotic device to provide new opportunities for exploratory learning and increased mobility.

Also, the present invention can be directed to active materials that are elastically stretchable and match the soft compliance of human skin and tissue (as opposed to conventional electronics and motors, which are rigid and inextensible). For example, a programmable orthotic device is described herein that can be intrinsically soft, wearable, pneumatically powered, and modular. When worn around a joint such as a wrist or knee, the orthotic will electronically monitor body motion and assist with motor function. In contrast to existing active orthotics (H. M. Herr and R. D. Kornbluh, “New horizons for orthotic and prosthetic technology: artificial muscle for ambulation,” in Proc. SPIE, vol. 5385, 2004, pp. 1-9; Y.-L. Park, B. Chen, D. Young, L. Stirling, R. J. Wood, E. Goldfield, and R. Nagpal, “Bio-inspired active soft orthotic device for ankle foot pathologies,” in Proc. IEEE/RSJ Int. Conf Intell. Rob. Syst., San Francisco, Calif., September 2011; L. Stirling, C. Yu, J. Miller, R. J. Wood, E. Goldfield, and R. Nagpal, “Applicability of shape memory alloy wire for an active, soft orthotic,” J. Mater. Eng. Perform., vol. 20, no. 4-5, pp. 658-662, 2011; H. Krebs, N. Hogan, W. Durfee, and H. Herr, “Rehabilitation robotics, orthotics, and prosthetics; chapter 48,” Textbook of Neural Repair and Rehabilitation (M. E. Selzer, S. Clarke, L. G. Cohen, P. W. Duncan, and F. H. Gage), Cambridge University Press, 2005) and exoskeletons (D. P. Ferris, J. M. Czerniecki, and B. Hannaford, “An ankle-foot orthosis powered by artificial peumatic muscles,” J. Appl. Biomech., vol. 21, pp. 189-197, 2005; K. E. Gordon, G. S. Sawicki, and D. P. Ferris, “Mechanical performance of artificial pneumatic muscles to power an ankle-foot orthosis,” J. Biomech., vol. 39, pp. 1832-1841, 2006), this device can be soft, modular, multifunctional, and composed almost entirely of elastomer and embedded micro-fluidic channels. This builds on work in soft robotics (Y.-L. Park, B. Chen, D. Young, L. Stirling, R. J. Wood, E. Goldfield, and R. Nagpal, “Bio-inspired active soft orthotic device for ankle foot pathologies,” in Proc. IEEE/RSJ Int. Conf. Intell. Rob. Syst., San Francisco, Calif., September 2011; C. Majidi, R. Kramer, and R. J. Wood, “A non-differential elastomer curvature sensor for softer-than-skin electronics,” Smart Mater. Struct., vol. 20, no. 10, p. 105017, 2011; K. Noda, E. Iwase, K. Matsumoto, and I. Shimoyama, “Stretchable liquid tactile sensor for robot joints,” in Proc. IEEE Int. Conf Rob. Autom., Anchorage, Ak., May 2010; Y.-L. Park, C. Majidi, R. Kramer, P. Berard, and R. J. Wood, “Hyperelastic pressure sensing with a liquid-embedded elastomer,” J. Micromech. Microeng., vol. 20, no. 12, 2010; Y.-L. Park, B. Chen, and R. J. Wood, “Design and fabrication of soft artificial skin with using embedded microchannels and liquid conductors,” IEEE Sens. J., vol. 12, no. 8, pp. 2711-2718, 2012) and embedded sensing (V. Giurgiutiu and A. N. Zagrai, “Embedded self-sensing piezoelectric wafer active sensors for structural health monitoring,” Trans. ASME, J. Vibr. Acoust., vol. 124, no. 1, pp. 116-125, 2002; A. Kadowaki, “Development of soft sensor exterior embedded with multi-axis deformable tactile sensor system,” in Proc. IEEE Int. Symp. Rob. Hum. Interact. Commun., Toyama, Japan, September 2009, pp. 1093-1098; Y.-L. Park, S. C. Ryu, R. J. Black, K. Chau, B. Moslehi, and M. R. Cutkosky, “Exoskeletal force-sensing end-effectors with embedded optical fiber-bragg-grating sensors,” IEEE Trans. Rob., vol. 25, no. 6, pp. 1319-1331, December 2009; Y.-L. Park, S. Elayaperumal, B. Daniel, S. C. Ryu, M. Shin, J. Savall, R. J. Black, B. Moslehi, and M. R. Cutkosky, “Real-time estimation of 3-d needle shape and deflection for mri-guided interventions,” IEEE/ASME Trans. Mechatron., vol. 15, no. 6, pp. 906-915, December 2010). These unique properties allow the soft orthotic to support a broad range of motor tasks and function reliably under extreme loads and body movements.

In one embodiment of the invention, a child can wear the second skin orthotic device during exploratory learning with one of several mechanical scaffolds specialized for each learning task. The second skin orthotic device can provide both resistance to motion and assistance to motion in order to encourage or cause new or additional movements during guided exploratory motion learning (e.g., spontaneous kicking). In addition, the second skin orthotic device can be programmed to adapt and evolve based on the progress of the subject.

To address the deficiencies of typical orthotic devices, systems and methods according to aspects of the present invention includes an actively controlled, wearable, modular orthotic device formed from an array of active components that can dynamically change the structural characteristics of the orthotic device according to programmed actuation of each of the modular elements which work together to produce assistive and resistive articulated motion of the corresponding body part, on which it is worn. Accordingly, the orthotic device according to aspects of the present invention can be effectively employed to provide articulation assistance or resistance of a body part, such as, a leg or arm or torso.

In one embodiment, an orthotic system includes a flexible skin like orthotic device formed from a flexible material and shaped to be worn over a body part. A plurality of active components can be embedded in the flexible skin and these components can cause the skin to actuate and become structurally modified in discrete areas forming a wearable scaffold. Each active component can include at least one controller adapted to control at least one actuator and at least one sensor coupled to the controller to enable the sensor to detect the motion of the actuator. Each of the active components can be connected with, at least one of, its adjacent active components, to allow information to be transmitted between active components to facilitate coordinated movement of the orthotic device. Further, each active component can be used to relay information from one adjacent active component to another adjacent active component. In this way, a communication network can be formed to enable the transmission of information between all active components of the network to facilitate controlled movement and shaping of the second skin orthotic device.

In another embodiment of the invention, an orthotic system includes: a garment formed from a flexible material and shaped to be worn over a body part; two or more one active components incorporated with the garment, wherein, in response to an actuation signal, the at least one active component changes state and causes the garment to become structurally modified; and a control system coupled to the two or more one active components, the control system being configured to provide different actuation signals to each active component over a period of use corresponding to a rehabilitation or therapy of the body part, the state of each active component being modified according to the different actuation signals, whereby the garment provides different levels of resistance and assistance or support to the body part over the period of use.

A further embodiment provides a method for operating an orthotic system, the orthotic system including a second skin orthotic device positioned over a body part, the orthotic device being formed from a flexible material, the method including: receiving, from at least one sensor coupled to the garment, information indicating an orientation of the body part; and in response to receiving the information from the at least one sensor, sending an actuation signal to at least one active component incorporated with the garment, wherein in response to an actuation signal, the at least one active component changes state and causes the garment to be structurally modified, whereby the modification of the garment encourages a change in the orientation of the body part or provides a different level of orthotic support to the body part.

Yet a further embodiment provides a method for operating an orthotic system, the orthotic system including a garment positioned over a body part, the garment being formed from a flexible material, the method including: receiving, from at least one sensor coupled to the garment, information indicating an orientation of the actuation component; and in response to receiving the information from the at least one sensor, sending different actuation signals to the at least one active component over a period of use corresponding to a motion of the active component, the state of the at least one active component being changed according to the different actuation signals, whereby the garment provides different levels of resistance and/or assistance or support to the body part over the period of use.

These and other aspects of the present invention will become more apparent from the following detailed description of the preferred embodiments of the present invention when viewed in conjunction with the accompanying drawings.

### BRIEF DESCRIPTION OF THE DRAWINGS

FIG. 1 shows a second skin orthotic device according to one embodiment of the present invention.

FIG. 2A shows a single actuation module according to one embodiment of the invention and FIG. 2B show that two or more actuation modules can be configured in an array in a layered sheet configuration according to an embodiment of the invention.

FIG. 3 shows each of the actuation modules connected by a flexible interconnect, according to aspects of the present invention.

FIG. 4A shows one embodiment of the second skin orthotic device control software architecture according to the invention and FIG. 4B shows an alternative embodiment of the second skin orthotic device control software architecture according to the invention.

FIG. 5 shows a clock driven scheduling approach that divides time into fixed width blocks, called rounds, according to an embodiment of the invention.

FIG. 6 shows that odd nodes and even nodes can follow matching schedules to communicate with each neighbor periodically according to one embodiment of the invention.

FIG. 7 illustrates how some embodiments of the present invention can be used to implement simple and complex motion applications.

FIGS. 8A-8F show miniature pneumatic muscle cells according to several embodiments of the invention.

FIG. 9 shows a design of the strain sensor according to one embodiment of the invention.

FIG. 10 shows flexible tube, second skin orthotic device according to one embodiment of the invention.

FIG. 11 shows an application of a second skin orthotic device according to one embodiment of the invention.

FIG. 12 shows a sequential contraction program application according to one embodiment of the invention.

FIG. 13A shows a robotic system according to one embodiment of the present invention. In this embodiment, the robotic system is adapted for use with a knee joint of a human patient. FIG. 13B shows an array of sixteen muscle/sensor units connected to an air source, where four of the sixteen muscle/sensor units are undergoing a contraction.

FIG. 14A shows a robotic system according to one embodiment of the present invention. FIG. 14C shows a pneumatic artificial muscle according to one embodiment of the present invention in a relaxed state (left side) and a contracted state (right side). FIG. 14B shows a side view of a robotic system according to one embodiment of the present invention, and FIG. 14D shows a close-up view of hyper-elastic strain sensor according to one embodiment of the present invention. FIG. 14E shows the testing of a hyper-elastic strain sensor according to one embodiment of the present invention while in a relaxed state (left side) and a stretched state (right side).

FIG. 15 shows four positions of a robotic system according to one embodiment of the present invention. In FIG. 15A, none of the embedded actuator/sensor modules is contracted. In FIG. 15B, all sixteen of the embedded actuator/sensor modules are contracted. In FIG. 15C, a first group of four of the sixteen of the embedded actuator/sensor modules are contracted. In FIG. 15D, a eight of the sixteen of the embedded actuator/sensor modules are contracted.

FIG. 16 shows five different configurations (FIGS. 16A-16E) of a robotic system according to one embodiment of the present invention where different combinations of embedded actuator/sensor modules are contracted to create different shapes.

FIG. 17A shows a controller software architecture according to one embodiment of the present invention. FIG. 17B shows a clock-driven scheduling approach according to one embodiment of the present invention.

FIG. 18A shows a test of an actuator according to one embodiment of the present invention. FIG. 18B shows a test of a strain sensor according to one embodiment of the present invention. FIG. 18C shows a test of an integrated strain sensor and actuator.

FIG. 19 shows test results from a robotic system according to one embodiment of the present invention subjected to six different air pressures between 250 and 550 kPa in 50 kPa increments.

FIG. 20A shows a calibration result for a strain sensor according to one embodiment of the present invention. FIG. 20B shows a calibration result for a sensor-actuator unit according to one embodiment of the present invention.

FIGS. 21A and 21B show a frame of reference, a geometry and a theoretical range of motion of a robotic system according to one embodiment of the present invention.

FIG. 22A shows the resistance through time for Case 1, where four muscles according to one embodiment of the present invention are simultaneously contracted. FIG. 22B shows the actual, estimated and desired angle change through time for Case 1.

FIG. 23A shows the resistance through time for Case 2, where the four muscles are contracted in sequence. FIG. 23B shows the actual, estimated and desired angle change through time for Case 2.

FIG. 24A shows the resistance through time for Case 3, where the first and third muscles are simultaneously contracted. FIG. 24B shows the actual, estimated and desired angle change through time for Case 3.

FIG. 25A shows the resistance through time for Case 4, where the second and fourth muscles are simultaneously contracted. FIG. 25B shows the actual, estimated and desired angle change through time for Case 4.

### DETAILED DESCRIPTION

The present invention is directed to an orthotic device formed from a plurality of active components and a method of controlling each of the active components to cause the orthotic device to move providing resistance and assistance to motion by a subject wearing the orthotic device. In accordance with the invention, the orthotic device includes an array of active components, each including at least one control element and the control element of at least two active components being in communication with each other to enable coordinated movements of the two active components.

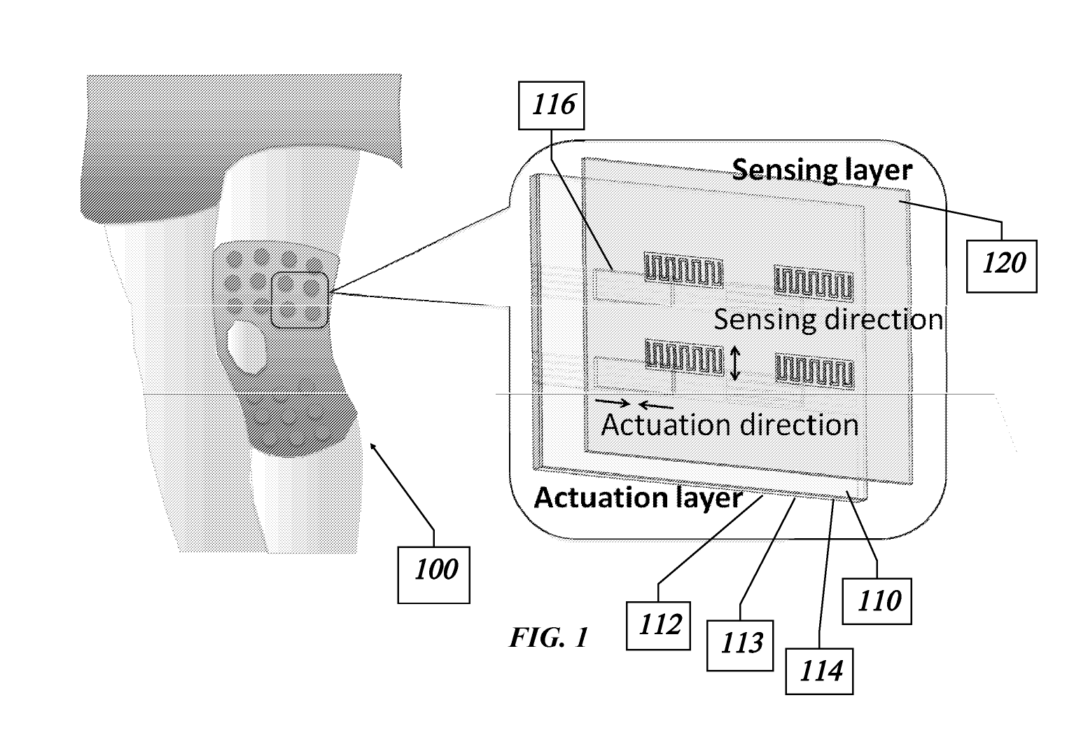

FIG. 1 shows a second skin orthotic device 100 according to one embodiment of the present invention. In this embodiment, the orthotic device 100 can be formed in layers and including an actuation layer 110 and a sensing layer 120 bonded to the actuation layer 110. The actuation layer 110 can include two or more layers of flexible material such as silicone or natural rubber selectively bonded together forming channels permitting the flow of fluids (including gases, such as air) and pockets connected to the channels which expand and contract as fluid is forced into or drawn out of the pockets. The actuation layer 110 can also include fibers embedded into the layer or adhered to the layer that constrain expansion of the pockets in an axial direction and allow expansion in other direction causing the pocket and the flexible material around the pocket to contract, forming an actuator. Each actuator can include a sensor, such as a strain sensor, bonded to the pocket of the actuator to provide an indication of the change in dimension of the actuator. Each actuator can further include one or more valves, such as a solenoid valve that controls the flow of fluid into or out of the actuator. Each actuator forms a pneumatically actuated muscle cell with a (strain) sensor.

The orthotic device 100 can be configured with two or more controllers, each controller being connected to one or more actuators to control the contraction and expansion of the actuators by controlling the flow of air (or other fluids) into and out of the actuator and receiving sensor information indicative of the contraction or expansion of the actuator. Each controller in combination with one or more actuators forms an actuation component or actuation module. Two or more actuation modules can be connected together to form an array of active components or modules that can selectively contract portions of the sheet orthotic device causing it to move and change its shape.

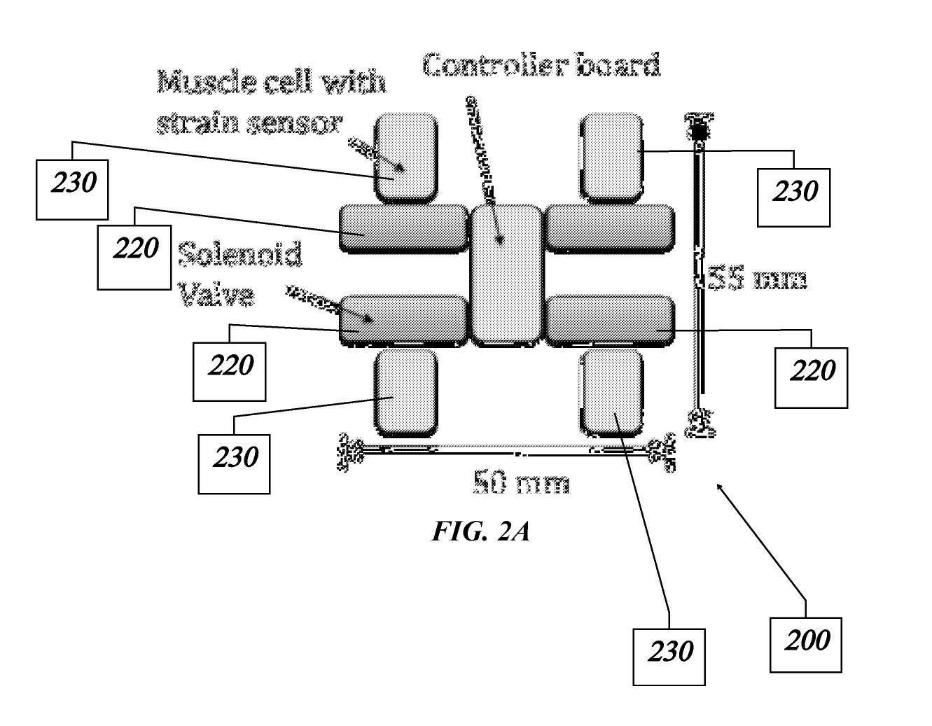

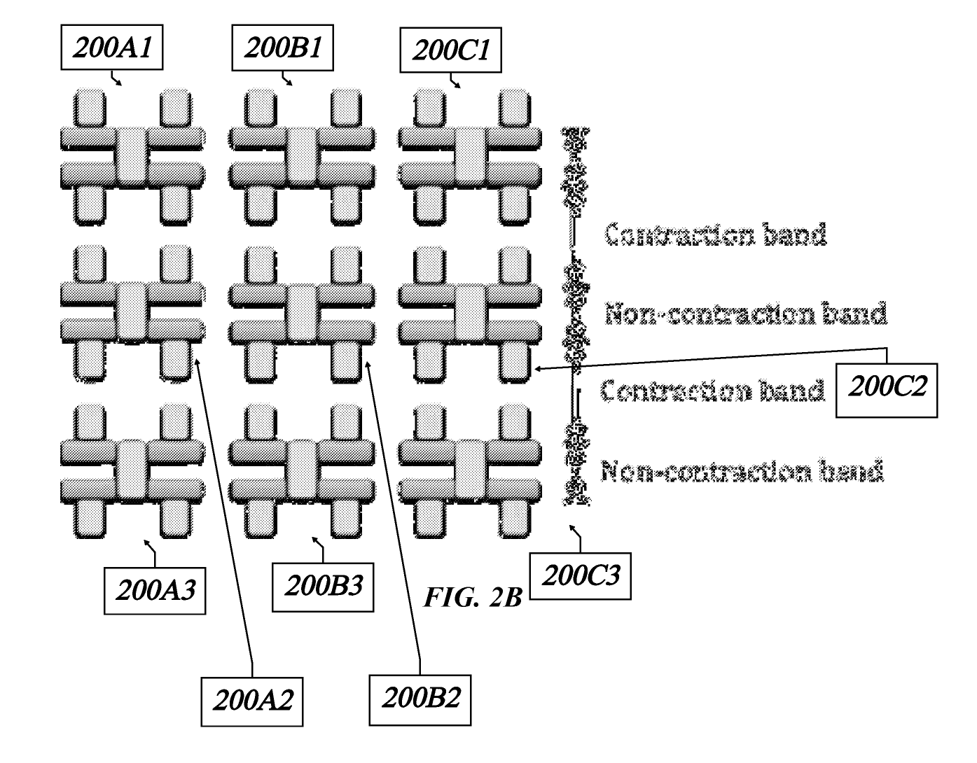

FIG. 2A shows a single actuation module 200 according to one embodiment of the invention. In this embodiment, one controller 210 can be connected to control four actuators (for example, muscle cells with associated strain sensors) 230. Four solenoid valves 220, controlled by the controller 210, can control the flow of fluid into or out of the four actuators 230. In other embodiments, one, two, or more actuators can be connected to the controller.

As shown in FIG. 2B, two or more actuation modules 200 can be configured in an array in a layered sheet configuration and controlled to provide complex structures and movements. For example, one row of actuation modules, such as 200A1, 200B1 and 200C1, can serve as a contraction band and can operate independently of or in a coordinated manner with another row of actuation modules, such as 200A2, 200B2 and 200C2, which can serve as a non-contraction band. Although nine actuation modules 200A1, 200B1, 200C1, 200A2, 200B2, 200C2, 200A3, 200B3 and 200C3 are shown with horizontal bands of coordinating actuators, the present invention includes any possible configuration of actuators operating individually or in coordination with one or more other actuators. Contraction and non-contraction actuators can be provided in bands, rows, in alternating patterns, or any suitable arrangement to facilitate complex structures and movements. For example, one or more actuators 200 can be controlled in a manner to resemble a muscle cell, a muscle fiber or strand, muscle tissue, tendon, and the like. Although contraction and non-contraction are described here, expansion and contraction functions can be performed by one or more actuation modules 200, as desired.

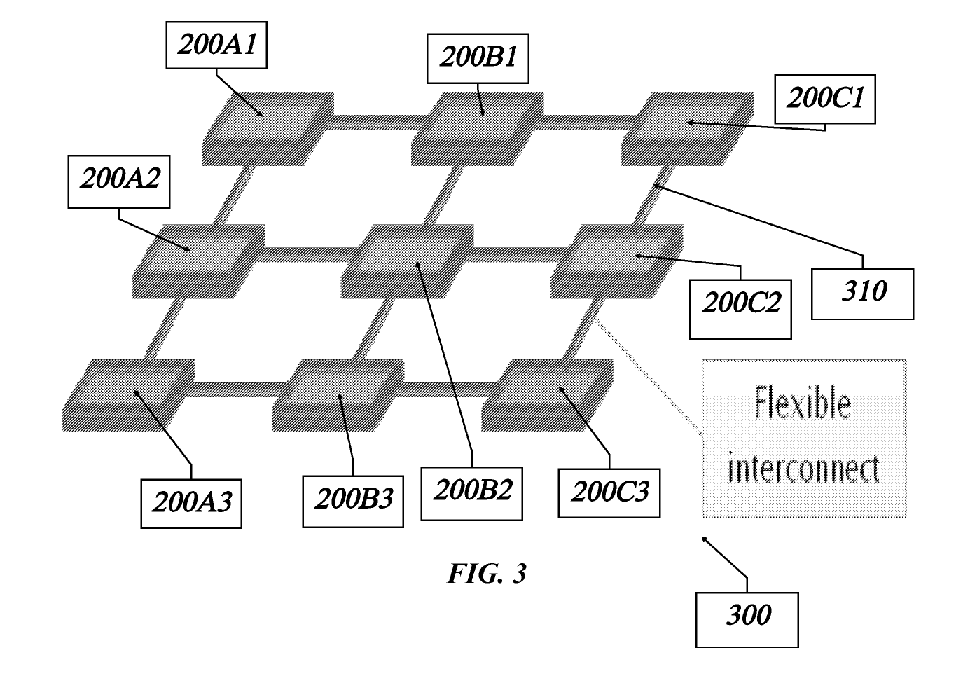

As shown in FIG. 3, each of the actuation modules 200A1, 200B1, 200C1, 200A2, 200B2, 200C2, 200A3, 200B3 and 200C3 can be connected, such as by a flexible interconnect (e.g. coiled wire) or a wireless interconnect (e.g., Blue Tooth, WiFi or, Zigbee) 310 to enable information to be transferred between modules forming a network of programmable embedded controllers 300. That is, the interconnect can be physical or wireless.

A network of programmable embedded controllers 300 can be used to control the sensor/actuator modules in the second skin orthotic device 100. The embedded controllers 300 can be connected in a two-dimensional mesh pattern, both spatially and logically, as shown in FIG. 3. Global motion patterns of the device can be carried out by scheduled (expansion and/or contraction) tasks that run on the network of controllers. In this topology, each controller, e.g., 200B2, can have four neighboring controllers, e.g., 200B1, 200A2, 200C2 and 200B3, that it can directly communicate with. The network forms the computation backbone that provides intelligent processing, interpretation, and timely detection of global patterns from real-time signals within the second skin. Based on the sensing results (sensed positions of the actuators), the controller network 300 can generate one or more actuation plans in response to certain global patterns. Optionally, the network 300 can also communicate to external devices to integrate with other computing infrastructure.

### Inter-Controller Communication and Synchronization

The most basic communication primitive provided by the controller network 300 can include inter-controller serial communication. Each pair of directly connected controllers, e.g., 200A1 and 200B1, in the mesh can communicate using a communication system. The communication system can comprise, for example, a two-wire serial protocol, such as RS-232, current loop, USB, Firewire or the like. Other, more advanced inter-controller communication can also be used. The controller network 300 may further comprise a power system for powering the communication systems. The power system for powering the communication system may comprise any suitable power storage or power delivery system. The power system can be external to the device or portable and incorporated into the device, which is desired. The power system may comprise a battery including disposable or rechargeable batteries.

The power system may, either alone or in combination with other power delivery systems, may comprise an energy harvesting system for converting kinetic energy (generated by the motion of a human or the device itself) to stored energy, which can be stored in a battery or directly applied to the device. The power generated by energy harvesting systems can be sufficient to power the communication system.

### System Software Architecture

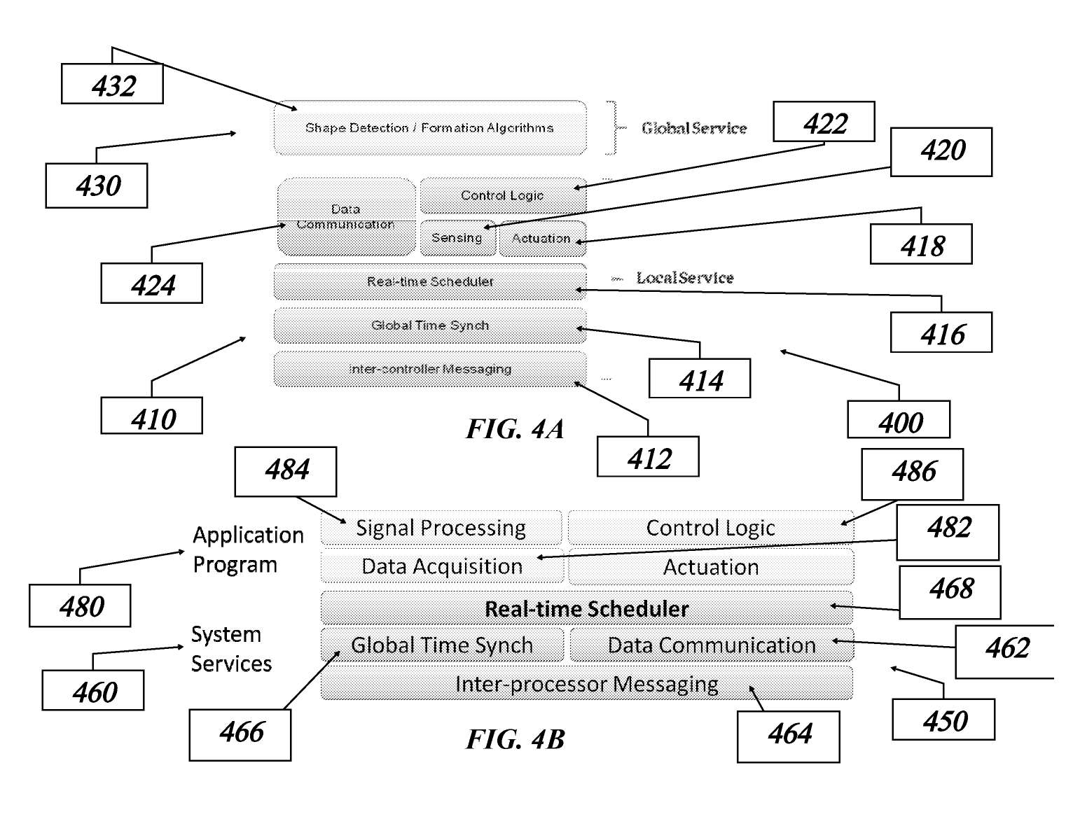

FIG. 4A shows one embodiment of the second skin orthotic device control software architecture 400. The system can be divided into two or more layers, including a local service layer 410, and a global service layer 450. The local service layer 410 can use fundamental software components or modules that manage local resources and provide high level primitives to support algorithms at the global service layer 450. Local service layer 410 can, for example, provide one or more services such as inter-controller messaging 412, global time synchronization 414, a clock-driven real-time scheduler 416, an actuation system 418, a sensing system 420, control logic 422 and a data communication system 424. The local service layer 410 can establish timing and handle inter-module communication with immediate neighboring modules, and issue commands to local actuators and receive information from sensors.

The global service layer 430 can be used to specify the application goal using the services provided by the local service layer 410. The local service layer 410 can be general purpose and can be shared among multiple functionally different applications of the second skin orthotic device 100, while the global service layer 430 can be customized for each application. Each application can be developed for assisting or resisting one or more predefined motions. The global service layer 430 can have, for example, a system for shape detection/formation algorithms 432.

FIG. 4B shows an alternative embodiment of the second skin orthotic device control software architecture 450. The system can be divided into two or more layers, including a system services service layer 460, and an application program service layer 480. The network of controllers can cooperatively carry out actions via known connectivity pattern. System services 460 can provide system primitives among controllers (data communication 462 and inter-controller (or inter-processor) messaging 464) and establish global time synchronization 466. The real-time scheduler 468 can be used to run tasks on time, for example, on time-slots. Control programs can be specified as scheduled tasks on multiple controllers. The application program layer 480 can provide control program services including data acquisition 482, signal processing 484, control logic 486 and actuation 488. The controllers can agree on a schedule for data communication and actuation.

### Clock Driven Scheduling

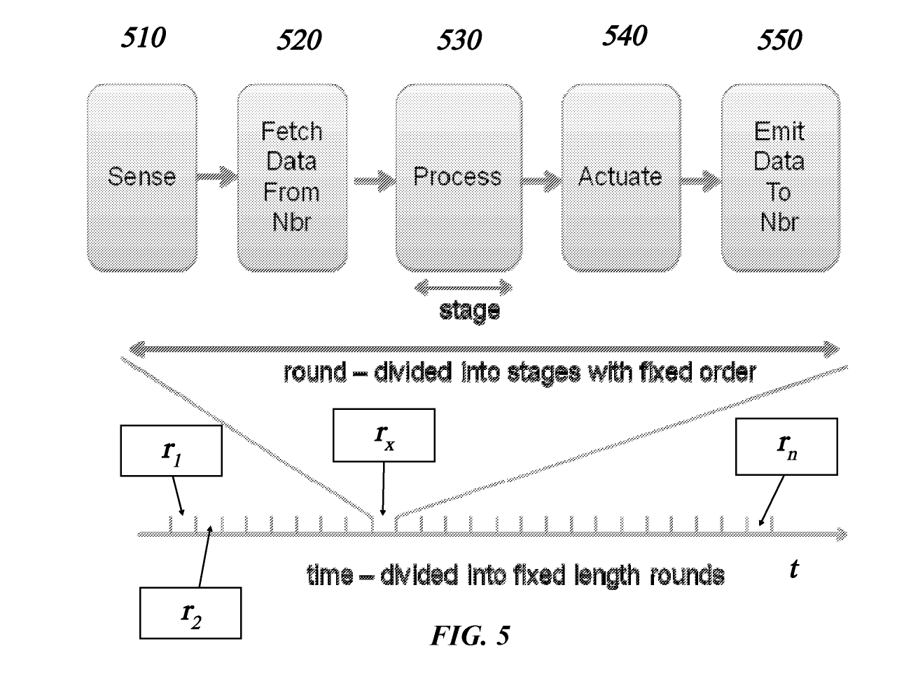

To provide a firm basis for coordinating controller behavior to achieve various motion and shape goals using the second skin orthotic device 100, the controller network 300 can be configured and constructed to provide predictable timing. For this purpose, a clock driven scheduling approach can be used to schedule software tasks at fixed time slots. As FIG. 5 shows, the system can divide time t into fixed width blocks, called rounds r₁, r₂, . . . , rₓ, . . . , rₙ. Each round can be further broken into five fixed-order stages or time-slots: Sense 510, Fetch 520, Process 530, Actuate 540, and Emit 550. All computational tasks for each controller can be abstracted into the five fixed-order stages. This basic structure for time division allows for the simplification of control on timing of sensing, processing, and actuation tasks. The lengths of each round and stage can be configurable and can be tuned to fit application specific goals.

### Scheduled Controller Communication

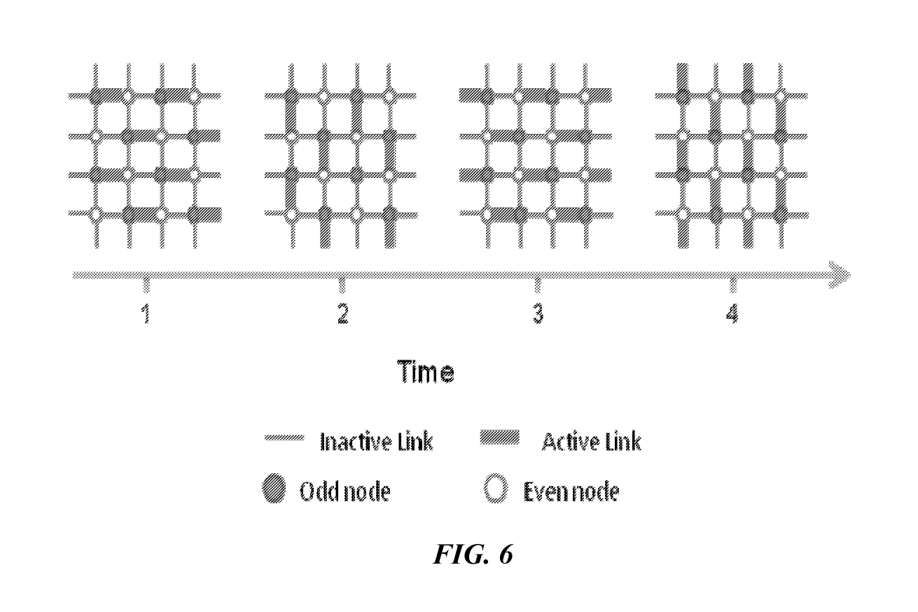

In accordance with one embodiment of the invention, each controller can be configured such that it can only communicate with one neighbor at any instant. In this embodiment, the controller network 300 can use a topology-aware scheduling scheme to carry out network-wide communication. The design can leverage the two-dimensional structure of the network. The controllers can be divided into two groups: odd nodes and even nodes. An odd node's neighbor can only be an even node and vice versa. As shown in FIG. 6, odd nodes and even nodes can follow matching schedules to communicate with each neighbor periodically. Unlike a centralized bus architecture, this architecture allows non-neighboring controllers to perform data communication in parallel.

### Global Control Program Structure

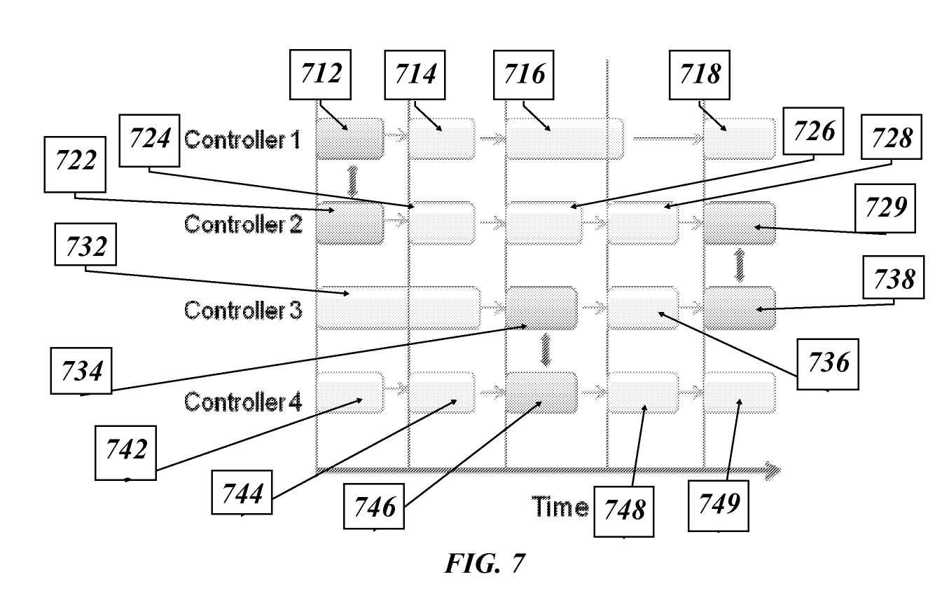

Enabled by the software architecture and the scheduling techniques provided above, the network of controllers 300 in the second skin orthotic device 100 can be used to implement many simple and complex motion applications by using scheduled tasks on multiple microcontrollers. FIG. 7 illustrates the concept. (Boxes 714, 716, 718, 724, 726, 728, 732, 736, 742, 744, 748 and 749 represent local processing tasks, and boxes interconnected by arrows 712/722, 729/738 and 734/746 represent communication tasks.)

### Shape Detection/Formation Algorithms

Leveraging the infrastructure designed above, the second skin device 100 can perform global shape detection and formation. This can be achieved by programming the controllers 300 to implement the following three algorithms at the global service layer:

Leader Election and Spanning Tree Formation Algorithm:
1. At the time of booting up, each controller obtains the numeric ID of each of its neighboring controllers. It assigns the neighbor that satisfies the two criteria its parent:
   a. The neighbor controller has an ID that is lower than the local controller's ID.
   b. The neighbor controller has the lowest ID among all 4 neighbors.
2. If a controller has no parent (i.e., the controller has the lowest ID among its neighbors), it assigns itself as the leader of the network.

The result of the above algorithm will form a spanning tree in the network that covers every node and rooted at the leader controller of the second skin network. Building on the leader election result, the following two algorithms provide the capability of detecting and forming a specific shape.

Shape Detection Algorithm:
1. Predefined shapes are stored on all controllers in the sensor mesh. The format of this knowledge is the desired strain sensor value at each second skin module.
2. Each controller samples strain sensor and determines the shapes it may be in.
3. Each module transmits (i.e., votes for) its potential shapes to its parent controller.
4. After Leader controller receives all votes. The global shape is determined by choosing the shape that has highest number of votes.

Shape Formation Algorithm:
1. Leader controller transmits the target shape to all controllers in the second skin device following the minimal spanning tree topology.
2. Each module sets target strain sensor values based on the target shape.
3. Each module runs a feedback controller locally to reach the desired sensor values.
4. Leader module determines if the target shape is reached using the shape detection algorithm.

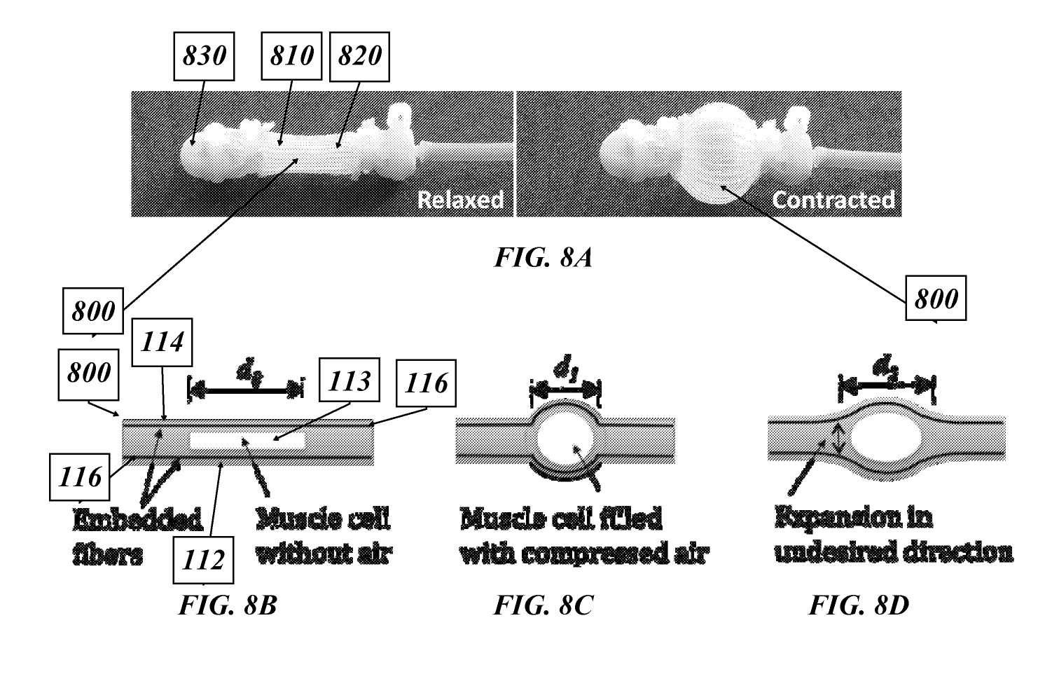

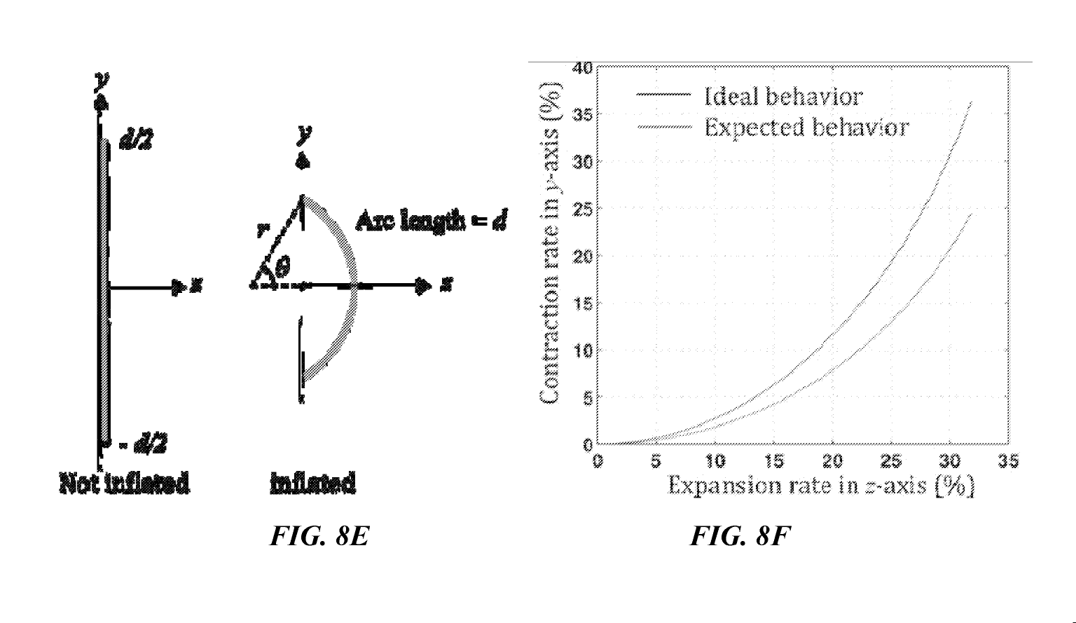

FIG. 8A shows miniature pneumatic muscle cells 800 according to one embodiment of the invention. Fixed-length Kevlar fibers 810 can be circumferentially embedded in a flexible polymer tube 820 that can be sealed at one end 830 and one or more tubes can be embedded in a polymer sheet. When pressurized air is introduced into the tube 820, the embedded fibers constrain expansion in an axial direction, but allow expansion in other directions, resulting in axial contraction (see, right side of FIG. 8A).

In accordance with an alternate embodiment of the invention, a pneumatically powered muscle cell 800 in a sheet configuration can be used as the main actuator. FIG. 1 shows the basic design of the actuator 110. A thin and narrow air chamber 113 can be embedded between two elastic layers 112, 114, and flexible but inextensible fibers 116, such as Kevlar threads, can be embedded in the two layers 112, 114 to constrain expansion resulting in axial contraction (see, right side of FIG. 8A and FIG. 8C). When pressurized air is introduced to the chamber from an external air source, such as an air canister or an air compressor, the embedded fibers 116 constrain the expansion in axial direction (y-axis) but allow the expansion of the chamber only in z-axis resulting in contraction in y-axis due to the fixed length of the fibers. FIGS. 8B, 8C, 8D describe the behavior of the muscle cell 800 with pressurized air supply. FIG. 8B shows the unexpanded length. The maximum contraction can be achieved when each layer makes a perfect semi-circular shape, as shown in FIG. 8C, and the theoretical maximum contraction rate can be simply calculated as:

$$\frac{Δ {l}_{max}}{{l}_{0}} = \frac{{l}_{0} - {l}_{min}}{{l}_{0}} = \frac{{l}_{0} - ( 2 {l}_{0} / π )}{{l}_{0}} = 1 - \frac{2}{π} = 36.3 \% \qquad ( 1 )$$

The actual contraction is expected to be smaller than the theoretical value, however, due to the inherent elasticity of the fibers and expansion of the rubber material between fibers in z-axis, as shown in FIG. 8D. Each muscle cell 800 is expected to be contracted up to approximately 25% with a maximum air pressure that the elastic layers 112, 114 can tolerate without being torn off.

The contraction rate of the muscle in y-axis is dependent on the expansion rate of the muscle cell in z-axis, which is dependent on the pressure of supplied air. FIG. 8E shows how the geometry changes when a muscle cell is inflated with air assuming the shape of the muscle cell with air is always part of a circle. If the initial length of the muscle cell without air is d, the half of the arc length of the inflated muscle cell is d/2=rθ. The expanded length in z-axis is z=r(1−cos θ), and the contracted length in y-axis is y=r sin θ. Then, the relationship between the contraction rate (c_{y}) in y-axis and the expansion rate in z-axis (e_{z}) can be found from the above three equations, as shown in FIG. 8F, where

$${c}_{y} = \frac{d - 2 y}{d} and$$

$${e}_{z} = \frac{d - z}{d} .$$

Each modular unit can be equipped with two strain sensors to measure the contraction of the muscle cell, embedded in the top and bottom layers each. FIG. 1 shows the configuration and placement of the strain sensors in accordance with one embodiment of the present invention. In operation, the muscle cell can experience much larger strain in the x-axis than in y-axis, the strain sensor can be placed perpendicular to the fiber axis (y-axis). When the muscle cell expands, the sensors can measure the strain changes of the top and/or the bottom layers during expansion. The measured strain values can be mapped with the displacement of the muscle cell and the actual contraction of the muscle cell can be acquired in real-time. This real-time sensor data can be highly useful to sense and control the overall orthotic device configuration.

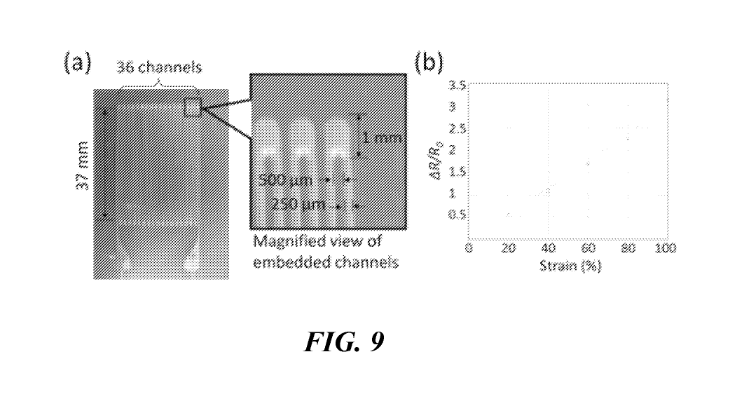

FIG. 9 shows a design of the strain sensor according to one embodiment of the invention. In this embodiment, micro-channels can be formed or embedded in a silicone rubber layer (EcoFlex0030), and the channels are filled with Eutectic Gallium Indium (EGaIn) liquid. Both the top and bottom layers can be cast on 3D printed molds and bonded using the same but uncured material. Only the bottom layer has the channels.

When the material experiences any strain in the axial direction of the channels, the overall channel length becomes longer and the cross-sectional area of channels becomes smaller which consequently increases the overall resistance of the channel.

In accordance with one embodiment of the invention, the channel size can be 250 μm (width)×250 μm (height). The nominal resistance of the device at rest can be approximately 5.8 ohm. FIG. 9 shows an example of the calibration result by applying axial strain up to 100%. The gauge factor (G) of a strain gage can be found from the following equation:

$$\frac{Δ R}{{R}_{0}} = G ɛ + α θ \qquad ( 2 )$$

where ΔR is the resistance change, R is resistance at rest, ε is applied strain, α is temperature coefficient, and θ is temperature change. Assuming there is no temperature effect, differently from normal strain gages, the gauge factor of this embodiment can be approximately 3.04 based on the calibration result.

The fabrication process of strain sensors can be similar to that of muscle cells except it requires liquid metal injection instead of fiber embedment. When the bonding process between the top and bottom layers is finished, the channels can be initially filled with air or another fluid, and the entrapped air (or fluid) can be replaced with EGaIn, as shown in FIG. 9.

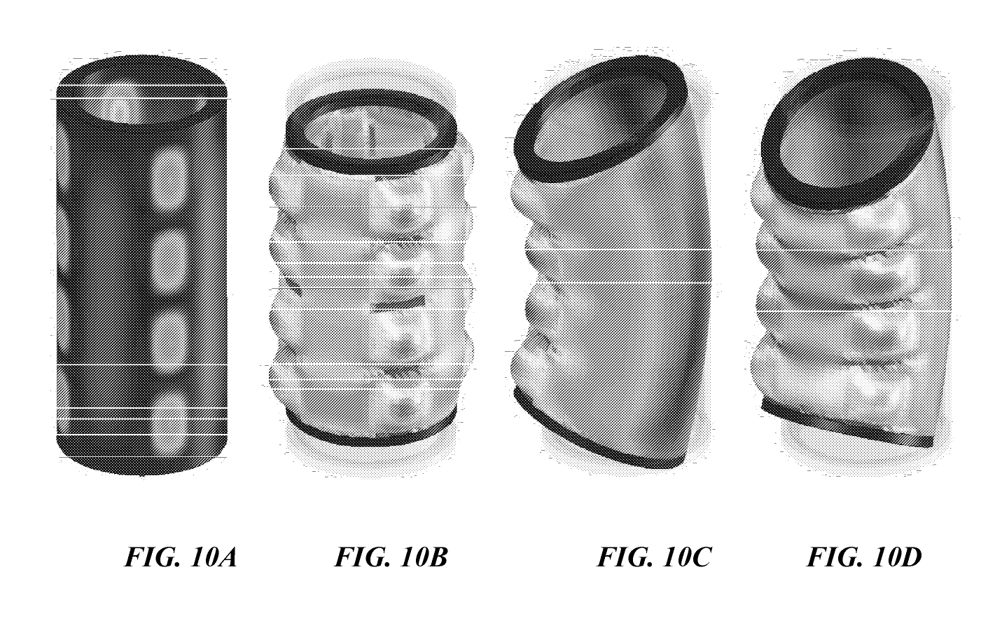

FIG. 10 shows flexible tube, second skin orthotic device according to one embodiment of the invention. In this embodiment, the orthotic device can include 16 actuator modules, each including one controller and 4 actuators. As shown in FIG. 10, when the pneumatic muscle cells are filled with air under program control of the controller, they expand outwardly and contract longitudinally along the axis of the tube. As shown in FIG. 10B, when all the actuators are contracted the tube shrinks in length. As shown in FIGS. 10C and 10D, when a select column of actuators are contracted the tube curls around the contracted actuators.

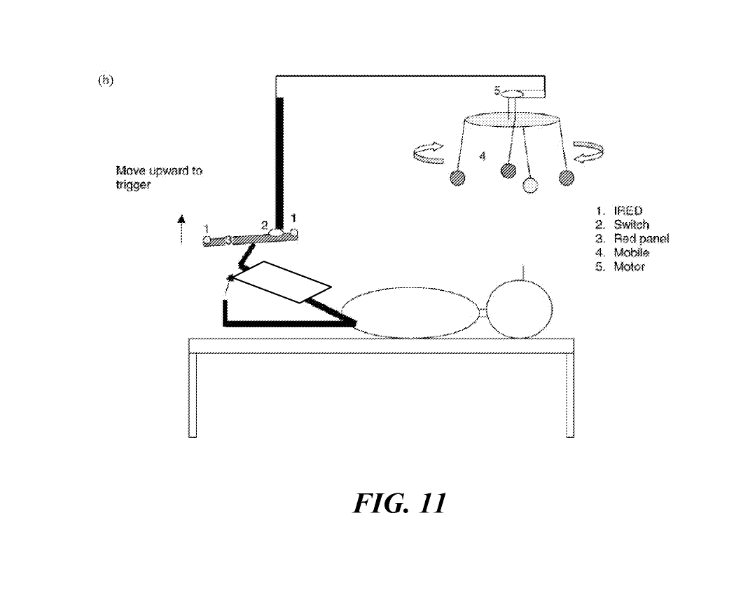

FIG. 11 shows an application of a second skin orthotic device according to one embodiment of the invention. In this embodiment, a second skin orthotic device such as that shown in FIG. 10 can be placed on one or both legs of an infant with prenatal brain injury. The sensors can be used to detect the motion of one or both of the subject's legs during exploratory learning and then can be programmed to assist and/or resist the exploratory learning motion to cause the subjects legs to move in preprogrammed motions. The programmed motions can be consistent with the motions produced by an infant of similar age or developmental character without brain injury so as to stimulate learning that might not be achieved due to the brain injury. These programmed learning tasks enable the subject to experience the environmental forces and tactile sensations that support development in normal subjects (those without brain injury).

In one embodiment, the second skin orthotic device according to one embodiment of the invention can be placed on one or more normal subjects (without brain injury) and the sensors can be monitored to record the motions of a normal subject. These motions can be stored on one of the controllers of the system so that then the system can be used by a subject with brain injury, the motion detected can be compared with the desired motion and the controllers can be activated to assist or resist the subject motion to induce further exploratory learning motion.

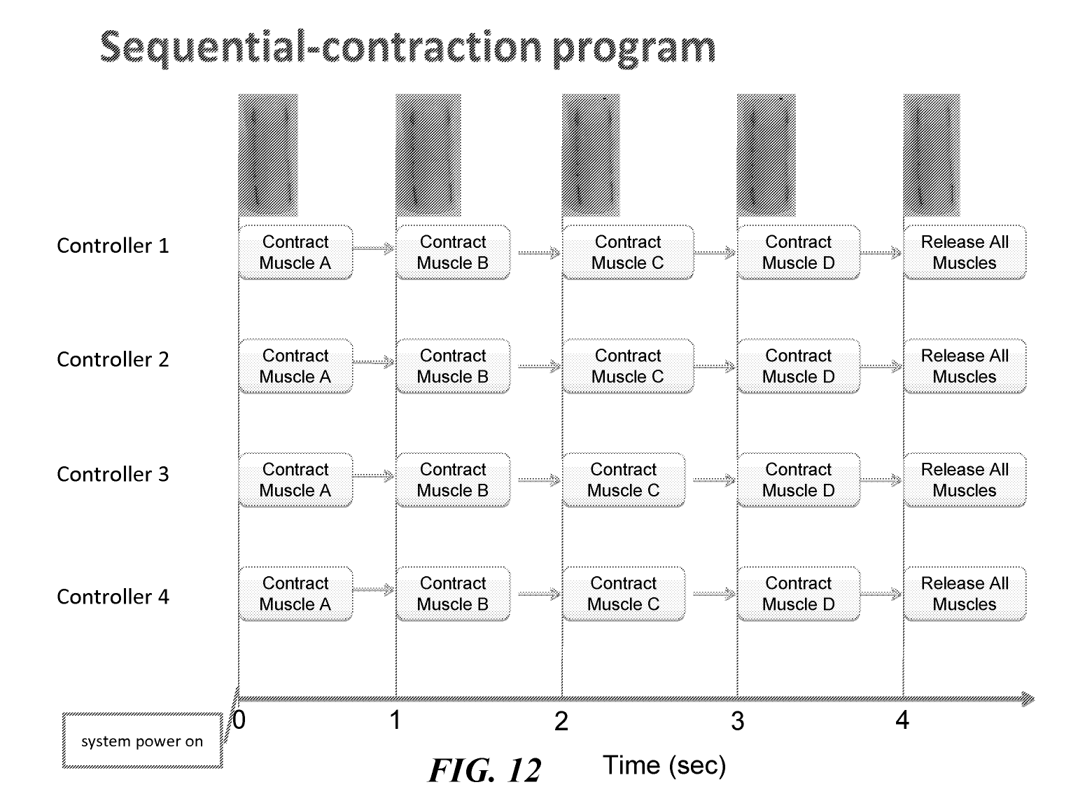

FIG. 12 shows a sequential contraction application program according to one embodiment of the invention. In accordance with this embodiment, the individual muscle cells of a flexible orthotic device according to the invention are sequentially contracted during the same round by each of the controllers. In the final round, all the muscles are released. The resulting motion would cause the tubular embodiment shown in FIG. 10 to shrink and then expand.

### Active Modular Elastomer Sleeve for Soft Wearable Assistance Robots

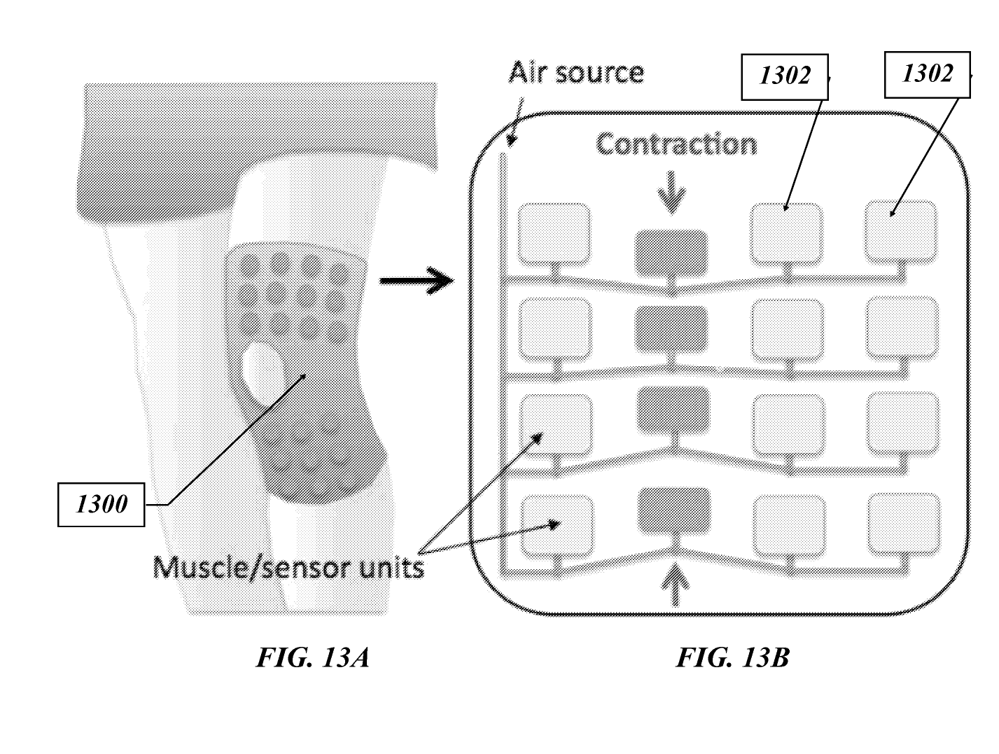

As illustrated in FIGS. 13A and 13B, a soft robot 1300 contains an array of independently controlled nodes 1302. Each node 1302 contains an actuator that controls rigidity and contraction, a sensor that monitors the output of the actuator, and digital circuitry for control and communication. When wrapped around a joint, this mesh of actuators and sensors can monitor and assist in both lower and upper body tasks.

In the present specification, we begin with an overview of a robotic device and fabrication including the node design, mesh topology, and software architecture (see, “DESIGN” below). In the “THEORY” section (below), we present the mathematical theories and design principles for the actuation and sensing elements of the soft orthotic. This is followed in the “CHARACTERIZATION” section (below) with the characterization of the robotic device comprised of sixteen independently controlled actuators. We close with a discussion (see, “DISCUSSION” below) and concluding remarks (see, “CONCLUSION” below) that review the current results, challenges, and outlook for programmable soft orthotics.

### Design

### Mechanical Design

The overall structure of the device can be a three-dimensional soft cylindrical sleeve (FIG. 13) with embedded soft pneumatic artificial muscles and strain sensors. The sleeve may be provided over a limb of a user. For example, FIG. 13A illustrates an example of a sleeve over a knee joint of a human user. When multiple muscles are actuated (contracted) (as shown, for example, FIG. 13B) collectively as a group, the overall displacement and force produced will be relatively high. Actuation may be achieved with any suitable means including a pneumatic system having an air source and connecting tubing attached to a plurality of muscle/sensor units 1302. The extremely flexible and elastic base material, a silicone elastomer, makes the robotic device conformable to the tapered cylindrical forms of human body segments. Each pneumatic muscle can be equipped with a soft strain sensor that detects the contraction of the pneumatic muscle. Four muscle cells with strain sensors are controlled by one micro-controller as one modular unit.

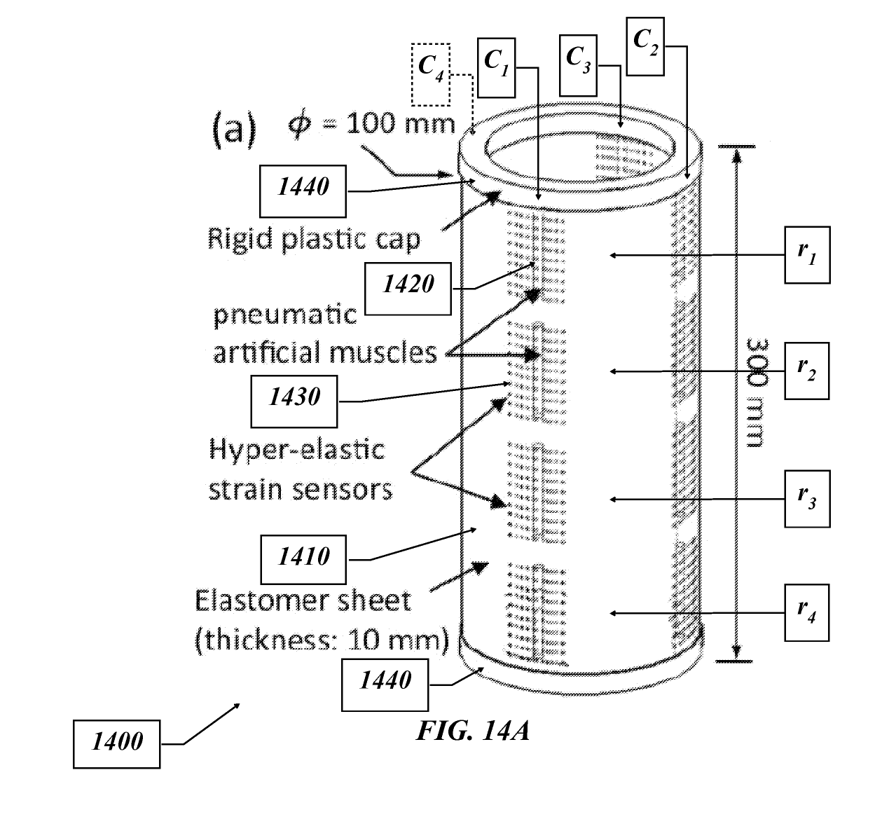

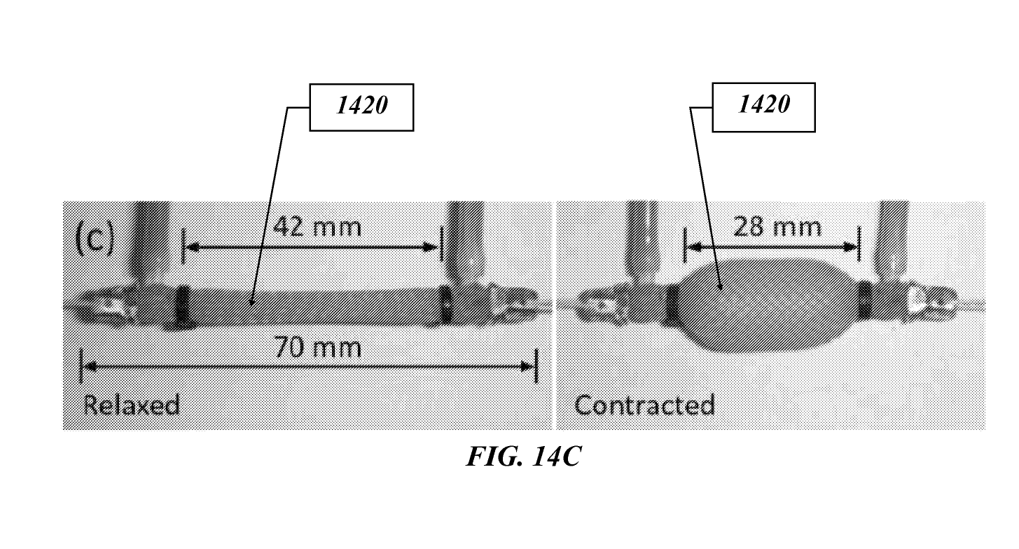

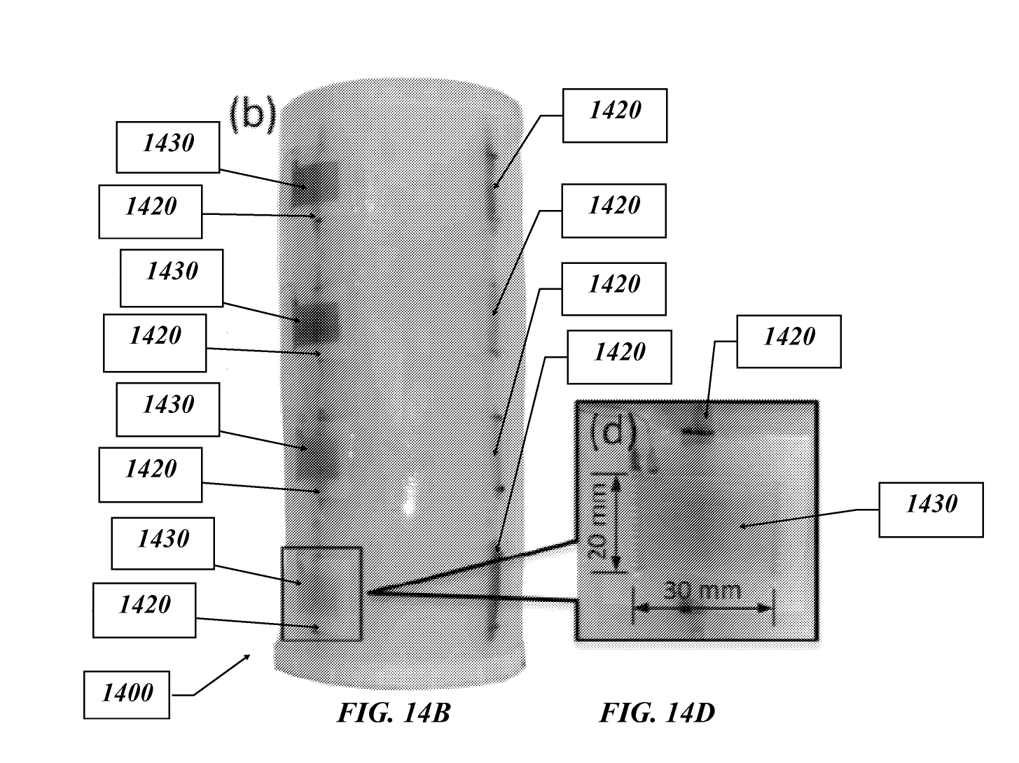

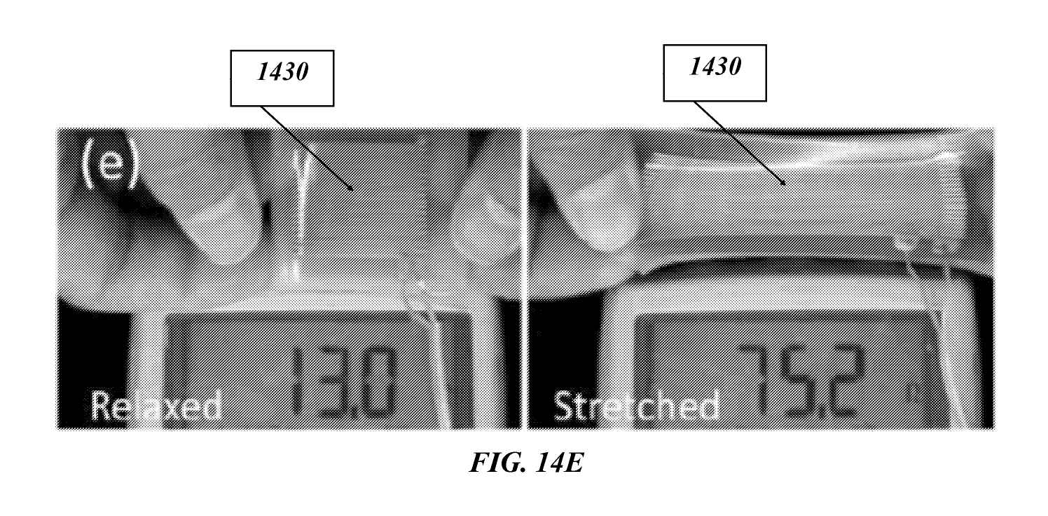

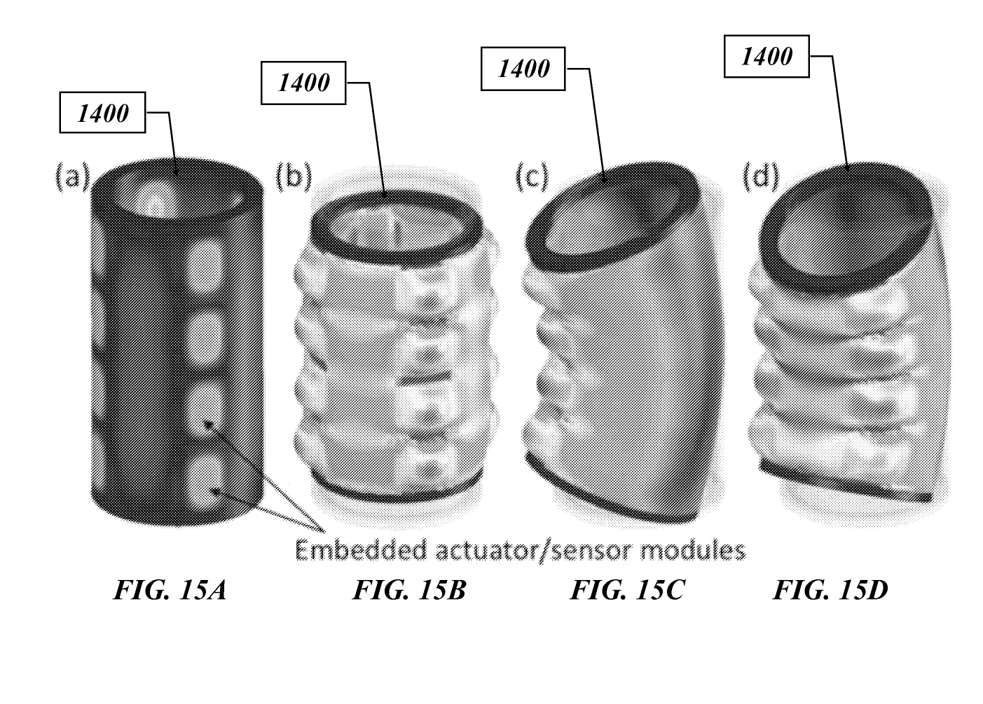

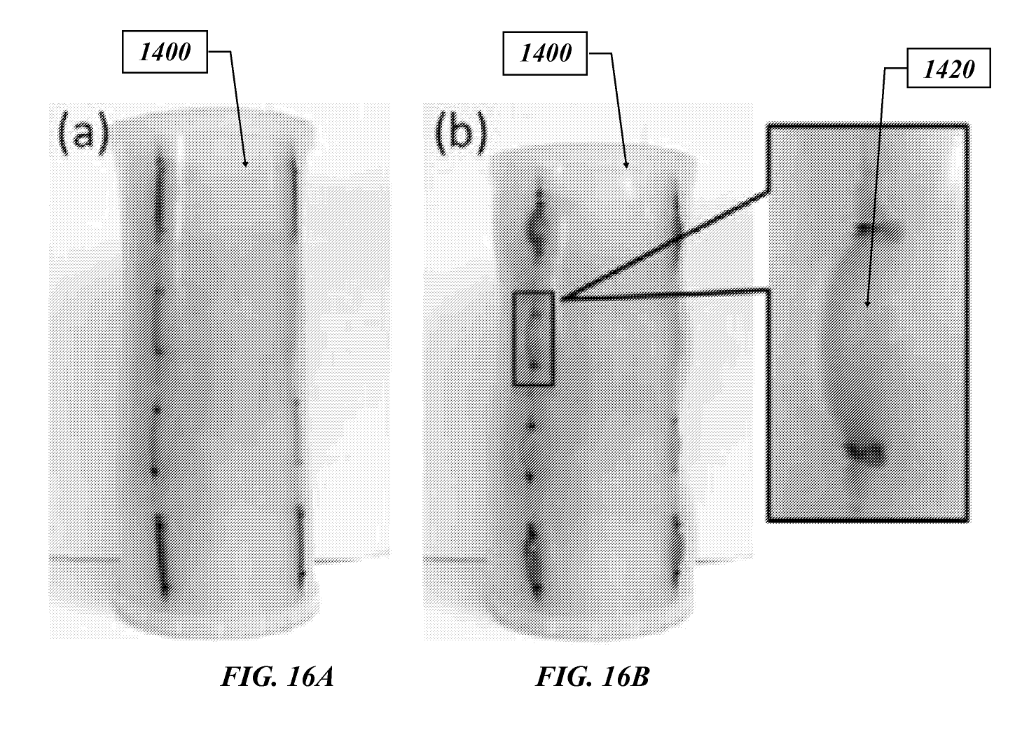

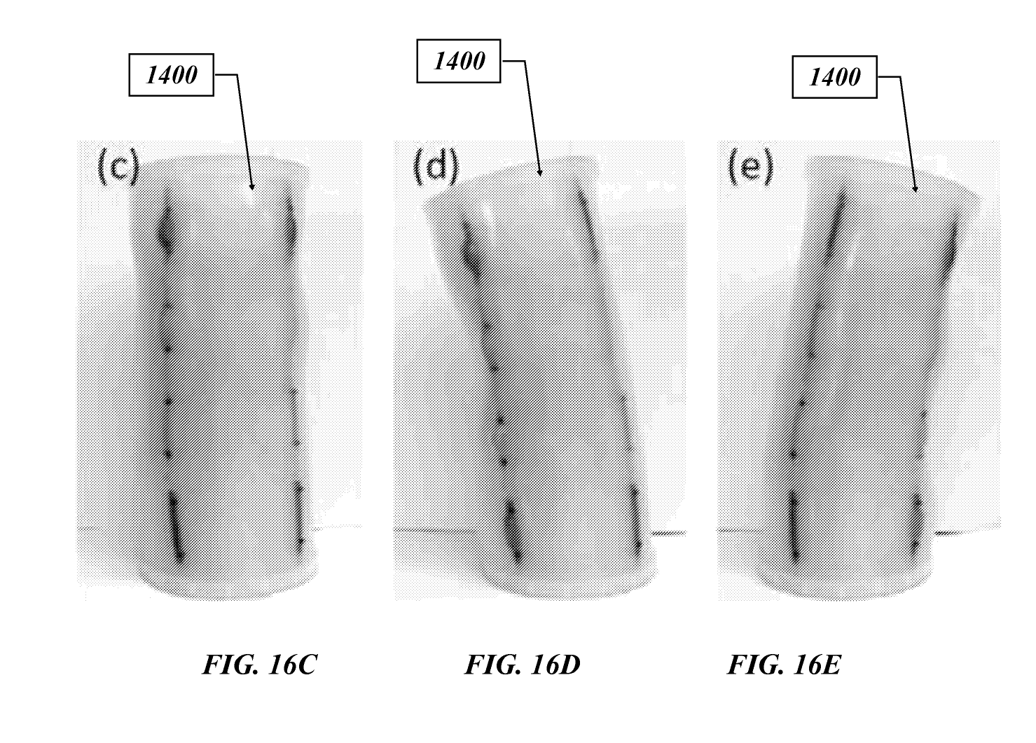

FIG. 14 shows a robotic device 1400 according to one embodiment of the present invention. The base material 1410 can be silicon elastomer (for example, EcoFlex 0030, Smooth-On, Inc., Easton, Pa. 18042, USA), and the actuators 1420 and sensors 1430 are embedded in the elastomer 1410. One or more rigid plastic caps 1440 can be used to constrain the ends of the base material 1410. One example of the device 1400 has four modular units with 16 muscles (4 rows r₁, r₂, r₃, r₄×4 columns c₁, c₂, c₃, c₄) in total. Each actuator 1420 can be connected to two off-the-shelf miniature solenoid valves (for example, NEX-2-03-L, Parker Hannifin Corp., Cleveland, Ohio 44124, USA) for compressed air injection and release, respectively. In an alternate embodiment, each actuator has its own strain sensor. In the illustrated embodiment, the strain sensors 1430 are integrated only in one actuator column c₁ (four actuators 1420) in the robotic device 1400. The weight of the robotic device 1400 can be approximately 1.2 kg but any suitable shape or size may be utilized. With different combinations of artificial muscle contractions, various shapes can be achieved, as shown in FIGS. 15A, 15B, 15C and 15D and FIGS. 16A, 16B, 16C, 16D and 16E.

FIGS. 16A, 16B, 16C, 16D and 16E are photographs of one embodiment of a robotic system according to the present invention. Here, the robotic system has an overall shape of a cylinder as described in greater detail elsewhere within the present specification. The cylinder comprises four combined sensor-actuator units arranged vertically into a single group. In this example, there are four groups of four combined sensor-actuator units, thus there are sixteen combined sensor-actuator units disposed inside the entire device. In FIG. 16A, all sixteen actuators are turned off. In FIG. 16B, all sixteen actuators are turned on resulting in a contraction, which is similar to a contraction of a natural human muscle. When the robotic system is contracted in this manner, the axial length of the robotic system is reduced. That is, the height of the cylinder in FIG. 16A is longer than the height of the cylinder in FIG. 16B. The inset of FIG. 16B is a close-up image of one of the actuators. The actuator bulges as compared to when the actuator is turned off.

In FIG. 16C, eight of the actuators at the top of the cylinder are turned on while eight of the actuators at the bottom of the cylinder are turned off, thus resulting in a vertical contraction of the upper portion of the system only. That is, the height of the upper half of the cylinder in FIG. 16C is longer than the height of the upper half of the cylinder in FIG. 16A (at rest); and the height of the lower half of the cylinder in FIG. 16C is about the same or exactly the same as the height of the lower half of the cylinder in FIG. 16A (at rest).

In FIG. 16D, eight of the actuators on the left side of the robotic system are turned on resulting in a bending of the robotic system to the left. In FIG. 16E, eight of the actuators on the right side of the robotic system are turned on resulting in a bending of the robotic system to the right. Although eight actuators are used to produce the bending motions shown in FIGS. 16D and 16E, any suitable number of actuators may be provided in the cylinder. Also, the sensor-actuator units need not necessarily be restricted to four units arranged vertically. That is, each combined sensor-actuator unit can be operated independently or in groups of two or more units.

The actuators can be powered by an external compressed air system or by a portable power means contained within or attached to the unit. The actuators can be powered by a self-contained pneumatic device, such as a cylinder of compressed air coupled with a regulator that releases air as desired. These cylinders can be relatively small, having a weight on the order of a few ounces each, and can provide relatively high pressure, that is, pressure sufficient to actuate the actuators of the present invention. Details associated with pneumatic actuation are disclosed, for example, by M. Wehner, Y.-L. Park, C. Walsh, R. Nagpal, R. J. Wood, T. Moore, and E. Goldfield, “Experimental characterization of components for active soft orthotics,” submitted in Proc. IEEE Int. Conf Biomed. Rob. Biomechatron., Roma, Italy, June 2012.

It is noted that the example of the robotic device shown, for example, in FIGS. 10A, 10B, 10C, 10D, 14A, 14B, 15A, 15B, 15C, 15D, 16A, 16B, 16C, 16D and 16E is not limited to a cylinder and may be provided in any desirable shape. For example, the robotic system can be adapted into a shape that is suited for a particular human body part such as a neck, spinal column, shoulder, elbow, wrist, finger joint, hip joint, knee, ankle, toe joint, individual muscle, muscle group and the like. The sensor-actuator units need not necessarily be provided in vertical groups of four. Overall design may be guided by the principle that actuators or groups of actuators function in a manner that is similar to one or more human muscles. The robotic system may be provided in a configuration that has an overall shape adapted to match that of the human body parts underlying the robotic system, and the actuators may be provided so as to perform in the same manner as the underlying human body part.

Also, the robotic system may have components provided in configurations that promote or facilitate a range of motion, a behavior, strength or resistance that is superior to natural or innate ability.

1) Actuation: Miniaturized McKibben-type pneumatic synthetic muscles 1420 were built and used as main actuators (FIG. 14C). A latex tube with air fittings was enclosed in an expandable mesh sleeve (for example, Flexo Pet, Techflex, Inc., Sparta, N.J. 07871 USA), and the ends of the mesh were clamped. When pressurized air is introduced to the latex tube through the air fitting, the mesh sleeve allows expansion in radial directions while creating contraction in its axial direction.

2) Sensing: Four pneumatic muscles 1420 are equipped with a hyper-elastic strain sensor 1430 (FIG. 14D) to measure the actuator contraction. The strain sensor 1430 consists of a micro-channel filled with liquid metal (EGaIn) that changes overall resistance of the channel as the sensor experiences strain changes (Y.-L. Park, B. Chen, D. Young, L. Stirling, R. J. Wood, E. Goldfield, and R. Nagpal, “Bio-inspired active soft orthotic device for ankle foot pathologies,” in Proc. IEEE/RSJ Int. Conf. Intell. Rob. Syst., San Francisco, Calif., September 2011; Y.-L. Park, C. Majidi, R. Kramer, P. Berard, and R. J. Wood, “Hyperelastic pressure sensing with a liquid-embedded elastomer,” J. Micromech. Microeng., vol. 20, no. 12, 2010; Y.-L. Park, B. Chen, and R. J. Wood, “Design and fabrication of soft artificial skin with using embedded microchannels and liquid conductors,” IEEE Sens. J., vol. 12, no. 8, pp. 2711-2718, 2012). Since each strain sensor 1430 is placed perpendicular to the axial direction of the corresponding pneumatic muscle 1420, the strain sensor 1430 detects the radial expansion of the actuator, as shown in FIG. 14A, and, the strain information needs to be converted to the contraction length of the muscle. FIG. 14E shows an example of a robotic device 1400 comprising a strain sensor 1430 in a relaxed state (left side of FIG. 14E) and the same robotic device 1400 comprising the strain sensor 1430 in a stretched state (right side of FIG. 14E).

### Programmable Controller Network

1) Controller Network Topology: A network of programmable micro-controllers (for example, Atmega1280, Atmel Corp., San Jose, Calif., USA) controls the soft orthotic modular units 1302 in the soft orthotic 1300. The controllers can be connected in a two-dimensional mesh pattern, both spatially and logically, as illustrated in FIG. 13. Global motion patterns of the device 1300 are carried out by scheduled tasks that run on the network of controllers. In this topology, each controller has four neighboring controllers that it can directly communicate with, using four dedicated serial communication ports. Therefore, the network of controllers provides the computation platform that hosts higher level intelligent processing, interpretation, and timely detection of global patterns from real-time signals within the orthotic 1300.

The controller network topology can be modified to suit the particular application of the device. For example, the orthotic or robotic system may be adapted for use for different body parts. Depending on the application, muscle groups of the device, represented, for example, by actuators or groups of actuators in a configuration such as that shown and described herein, are combined in different ways to achieve different behaviors, and the behaviors of the muscle units or groups are managed by the present software. For example, for an application of the device to the human knee, the actuators within the device are programmed to work together, in one embodiment, to keep a human patient's knee from bending (for example, if the human patient's knee is injured or recently operated upon and where motion is undesirable). In another embodiment, the actuators within the device may be programmed to allow or promote desired movements. For example, the device can be programmed to move and then stop at a desired point.

The device may comprise sensors to detect the motion of the device itself and a second set of sensors to detect the motion of a human body part, for example, a knee. The present invention includes software programmed to receive and process information from one or more groups of sensors. One example of a type of sensor used with the present device is an inertial measurement unit (IMU), which utilizes an accelerometer and a gyroscope. The present software may be adapted to receive information from a sensor, such as an IMU, to specify, for example, an angle of a human body part, such as the angle between the lower leg and the upper leg as measured at the human knee. That is, the IMU provides the system with information regarding the behavior and motion of the human knee, which can be used to send instructions to the controller of the device (and thus the actuators) as to what the device needs to do. This information can change based on information from the IMU regarding the status of the human body part in question.

For example, a human patient has an injured knee. The human patient undergoes an operation to facilitate restoration of the knee's function. After surgery, the human patient undergoes rehabilitation on the knee. The rehabilitation can employ use of the present device to facilitate restoration of the knee's function. The device can be adapted to sense information about the natural motion of the knee, which may, at first, be impaired. As rehabilitation and healing progress, the device can utilize information from a sensing device, such as an IMU, to measure and analyze change in the natural function of the knee. The present software can adjust programmed actuation patterns of the device to account for changes in the human's condition. For example, in the beginning of a rehabilitation process, the device may initially be programmed to provide more assistance to the human in performing a natural function, for example, bending of the knee and support of the body during walking. The present software might initially sense weakness in the natural body part resulting in a strengthening of the orthotic by engaging the orthotic device's actuators. Over time, as the human patient heals and natural strength and function are restored, the present software may dynamically adjust the amount of assistance required by the device to facilitate the human patient's natural healing. Also, during healing, certain configurations of the human patient's body part may be undesirable during one phase of the rehabilitation process, and the software can be programmed to restrict such motions. For example, a doctor may order a limited range of motion immediately after surgery. The device can be programmed to restrict movement beyond certain set parameters. During this time period, if the human user attempts to move the joint beyond an acceptable range or if an external force causes the joint to move beyond a pre-specified tolerance, the device can be programmed to actuate in a manner that resists such motion, thus promoting desirable behaviors and, in this example, healing. Another example relates to providing infants with enriched opportunities for guided exploratory learning, described in greater detail above.

The communication system can comprise an advanced processor. For example, wireless systems, which may include radio technology, are part of the wireless system and are preferably powered in a self-contained manner. The present software may be adapted to provide power to the communication system only as needed instead of being powered at all times.

The controller network topology can facilitate distributed control. For example, in some embodiments, some units of the device may not require participation at a given point in time. By turning some units off during operation, power can be conserved.

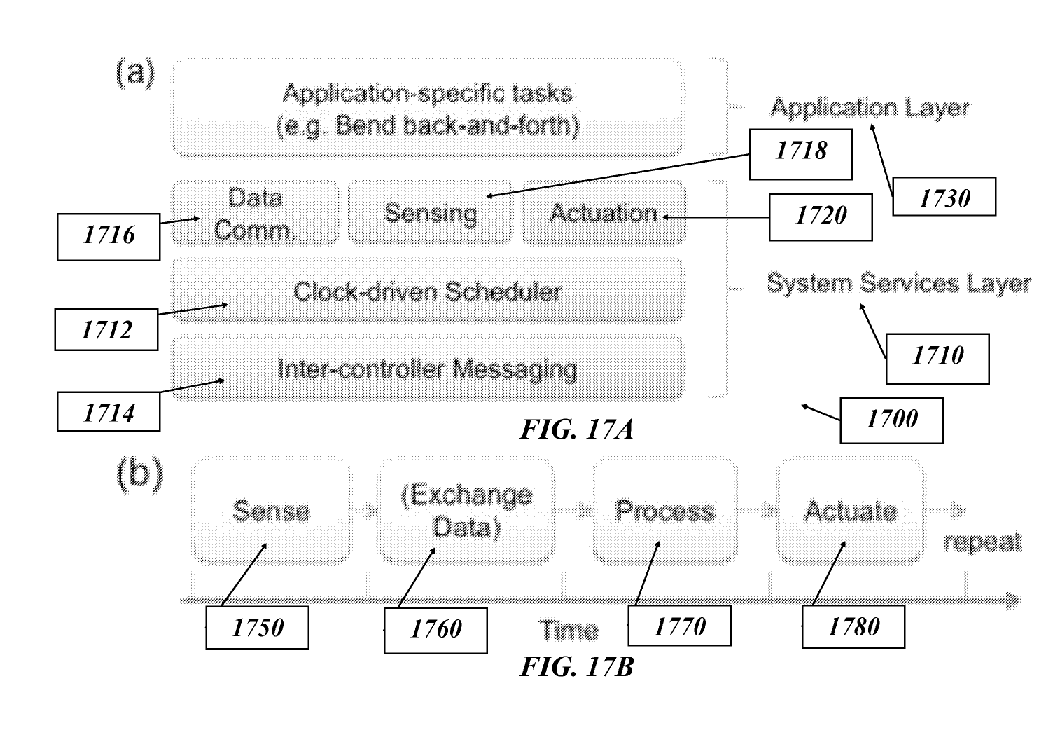

2) System Software Architecture: FIG. 17A shows the controller software architecture 1700. The architecture can be divided into two main layers: System Services Layer 1710, and Application Layer 1730. The System Services Layer 1710 implements fundamental components that manage local resources and provide primitives to support algorithms at the Application Layer 1730. The System Services Layer 1710 implements a clock-driven scheduler 1712, handles inter-module (or inter-controller) messaging 1714 and/or data communication 1716 with neighboring modules 1714, processes readings from strain sensors with a sensing system 1718, and sets actuation parameters through an actuation system 1720. The Application Layer 1730 specifies the application goal using the services provided by the System Services Layer 1710.

The software may be programmed to include consideration of power requirements of components of the device including, for example, a motherboard, a battery, an actuator, a sensor, a wireless device and the like.

3) Clock-driven scheduling: A clock-driven scheduling approach can be used to schedule software tasks at fixed time slots to provide predictable execution of specific tasks at individual modular units. As FIG. 17B shows, all computational tasks are scheduled to run at fixed schedules. Such structure allows simplification of control on timing of sensing 1750, processing 1770, and actuation 1780 tasks. An exchange data task 1760 can be performed if and as needed, for example, after the sensing task 1750 and before the processing task 1770. At the Application Layer 1730, the timing of tasks can be configurable to fit application specific goals.

4) Global Control Program Structure: Leveraging the services outlined above, the orthotic device can be programmed to perform desired motion patterns. The motions are described as scheduled tasks on multiple microcontrollers.

### Fabrication

After preparing all the components, such as sensors and actuators, they are integrated into a device that is shown in FIG. 14B. The fabrication process can be divided into three steps.

The first step can be to cast a flat elastomer sheet 1410 with embedded actuators 1420 and sensors 1430. In this step, four actuators 1420 are tied in series through Kevlar fibers, and four actuator groups (along column c₁) are placed in parallel in a flat mold. Then, liquid polymer can be poured. The strain sensors 1430 are connected through thin and flexible copper wires that ensure the electrical connection during different motions of the device 1400.

The second step can be to make the cured elastomer sheet a cylindrical shape such as that shown in FIGS. 14A and 14B. The flat elastomer sheet can be curled and placed in a semicylindrical mold, and small amount of liquid polymer can be poured inside of the curled elastomer cylinder to make a seam.

The final step can be to assemble the plastic caps 1440 to the both ends of the cylinder. The Kevlar fibers from the end of each actuator column c₁, c₂, c₃ and c₄ are fixed to the cap 1440 to ensure the desired motions of the cylinder with contraction.

Since each actuator 1420 can be equipped with its own solenoid valves, it can be individually controlled for contraction and release.

FIG. 14D shows a close-up view of an example of a combined sensor-actuator unit. FIG. 14B shows an example of four combined sensor-actuator units arranged vertically into a single group. In this example, there are four groups of four combined sensor-actuator units, thus there are sixteen combined sensor-actuator units disposed inside the entire device. In this example, the four combined sensor-actuator units may be powered by a shared, self-contained power system, such as a single battery.

### Theory

In order to monitor and assist human joint motion, the soft orthotic 1300 must be designed to register strains and produce forces that are comparable to those of natural muscle. Design can be based on principles of mechanics, which govern the strain response, power output, and rigidity of the modular elements.

We tune these properties by selecting the appropriate materials, geometries, and operating pressures.

### Pneumatic Artificial Muscle Actuator

Each node of the modular array contains a McKibben-type pneumatic actuator that controls both the natural length and rigidity of the node. The actuator can be composed of an inextensible thread of length b wrapped around a rubber cylinder that has a natural length L₀, radius R₀, and elastic modulus E. The thread has a pitch θ₀=cos⁻¹(L₀/b) and is wrapped n=b sin(θ₀)/2πR₀ times around the cylinder (C.-P. Chou and B. Hannaford, “Measurement and modeling of mckibben pneumatic artificial muscles,” IEEE Trans. Rob. Autom., vol. 12, no. 1, pp. 90-102, February 1996). Since b and n are both fixed, the kinematics of the actuator are restricted to a single free parameter: the length L of the actuator at static equilibrium.

Under a tensile load F, the total potential energy of the actuator has the form

$$\begin{matrix}U = U ( L ) = - P π {P}^{2} L - FL + \frac{π EhRL}{1 - v} \{ {ɛ}_{ψ}^{2} + {ɛ}_{n}^{2} + 2 v {ɛ}_{ψ} {ɛ}_{ψ} \} \\ where & ( 1 ) \\ R = \sqrt{\frac{{b}^{2} - {L}^{2}}{2 π n}} , {ɛ}_{ψ} = \frac{{R}^{2} - {R}_{0}^{2}}{2 {R}_{0}^{2}} , {ɛ}_{n} = \frac{{L}^{2} - {L}_{0}^{2}}{2 {L}_{0}^{2}} , & ( 2 )\end{matrix}$$

h is the thickness of the cylindrical wall, and v=½ is its Poissons ratio. At equilibrium, U must be stationary with respect to L such that dU/dL=0. However, determining L at equilibrium requires solving a sixth order polynomial. Analysis is simplified by ignoring the elastic contribution of the rubber shell, i.e., U=−PπR²L−FL. In the absence of a tensile force F (i.e., F=0), the equilibrium length of the actuator becomes L*=0.577b for any value of P>0.

Filling the actuator with a pressure P>0 will cause the actuator to shorten from a length L₀ to a final length

$$L = \sqrt{\frac{1}{3} ( \frac{2 n F}{P} + {b}^{2} )} . \qquad ( 3 )$$

Suppose, for example, that the actuator is placed adjacent to a joint of radius λ. Inflating the actuator with a pressure P will cause the joint to rotate by an amount Δθ=(L₀−L)/λ. Here, F is replaced with M/λ, where M is the magnitude of applied torque. In the absence of an external torque, the joint will rotate by (L₀−0.577b)/λ radians. From this new reference state, the torsional spring constant of the joint is κ=1.732Pbλ2/n.

For N actuators in parallel, the angle of rotation during contraction remains the same but the torsional spring constant (and torque required to prevent joint rotation) increases by a factor of N. In contrast, for N actuators in series, the angle or rotation increases by a factor of N but torsional rigidity reduces by a factor of N.

### Strain Sensor

Joint motion and actuator contraction are monitored with strain sensors that are embedded in the host elastomer. The design of these sensors can be based on the Whitney strain gauge and are composed of a rubber tube filled with conductive liquid (R. J. Whitney, “The measurement of changes in human limb-volume by means of a mercury-in-rubber strain gage,” Proc. Physiol. Soc, vol. 109, pp. 5-6, 1049). As the tube stretches with a strain ε, the cross-section decreases and the electric resistance increases by a relative amount 2ε+ε². For a strain sensor of length ζ placed around a joint of radius λ, a bending angle Δθ corresponds to a relative change in resistance of

$$\frac{Δ R}{R} = \frac{2 λ}{ζ} Δ θ \qquad ( 4 )$$

If, for example, ζ=0.1λ, then a 10° bending angle results in a 3.4% increase in electric resistance.

### Elastomer Design

In order to avoid interference with the body mechanics, the elastomer should exhibit approximately the same mechanical compliance as natural skin. The membrane stiffness of the elastomer is S=EH, where E is the elastic modulus and H is the elastomer thickness. Therefore, E and H must satisfy the condition EH=EₛHₛ, where Eₛ and Hₛ are the modulus and thickness of natural skin, respectively. Hence, an elastomer with an elastic modulus on the order of skin (1 MPa) must share the same millimeter thickness as the epidermis.

Alternatively, elastomer stiffness may be represented in terms of the corresponding stiffness of a torsional spring. As the joint bends, the orthotic stretches and exerts a torque about the center of the joint. For a single module unit of thickness H, width w, and length ζ, the torsional spring constant will be κ=EwHλ2/Θ, where λ is the joint radius. For a soft-as-skin orthotic, this torsional spring constant will be small compared to the natural joint stiffness. This assumption is reasonable since even natural skin has little influence on joint stiffness, which is instead governed by the elasticity of muscles, tendons, and subcutaneous tissue.

### Characterization

The robotic device 1400 was characterized in two different levels. The individual components, actuators and sensors, were first characterized, and then, the integrated device that has 16 embedded sensor-actuator units was characterized.

### Actuator

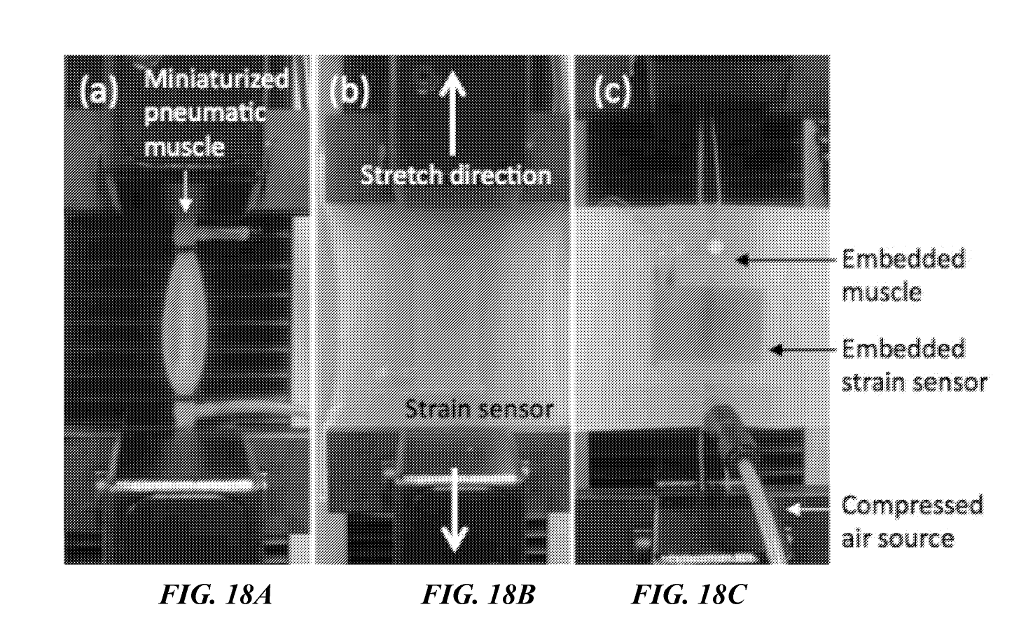

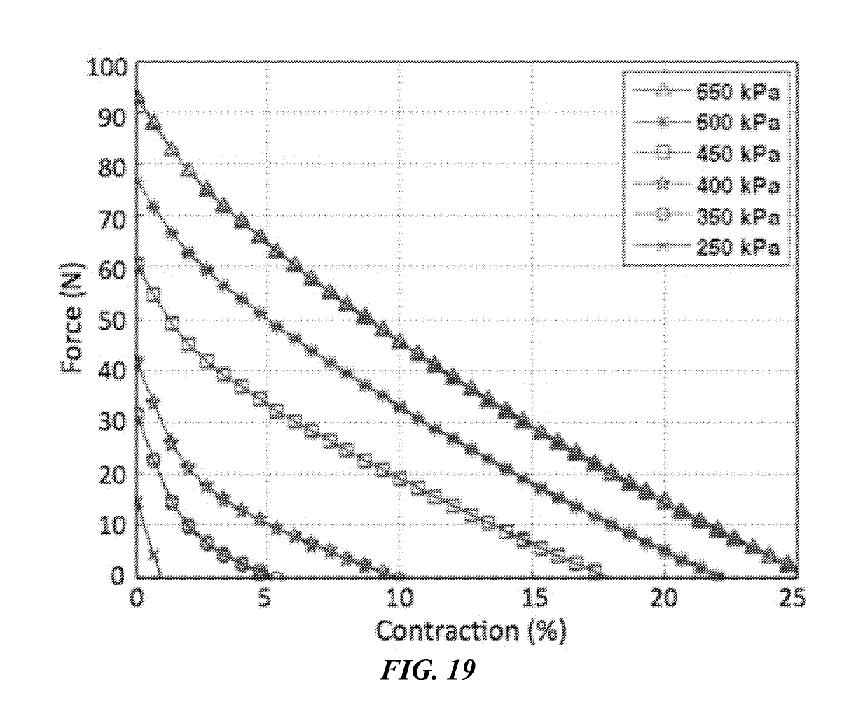

The custom-built pneumatic artificial muscle was experimentally characterized. The McKibben pneumatic actuator shows different behaviors for different contraction rates and air pressures. A single actuator was fixed to a commercial materials tester (for example, Instron 5544A, Instron, Norwood, Mass. 02062, USA), as shown in FIG. 18A, and a constant air pressure was provided while maintaining the original muscle length. Then, the artificial muscle was gradually released at a rate of 2 mm/sec until the axial contraction force reached to 0. By repeating this experiment with varied air pressures, the contraction forces were measured with different contraction rates. The full characterization result is shown in FIG. 19.

When the air pressure was increased up to 550 kPa, the contraction rate increased up to 25% in the experiments. The maximum achievable force was approximately 93 N with a pressure of 550 kPa. More detailed characterization results can be found in M. Wehner, Y.-L. Park, C. Walsh, R. Nagpal, R. J. Wood, T. Moore, and E. Goldfield, “Experimental characterization of components for active soft orthotics,” submitted in Proc. IEEE Int. Conf Biomed. Rob. Biomechatron., Roma, Italy, June 2012.

Since the pneumatic muscle produces its maximum force at the start of contraction, it will be useful to initiate the joint motion when it cooperates with the user's biological muscles because biological muscles have minimum contraction force when they are fully stretched.

### Sensor

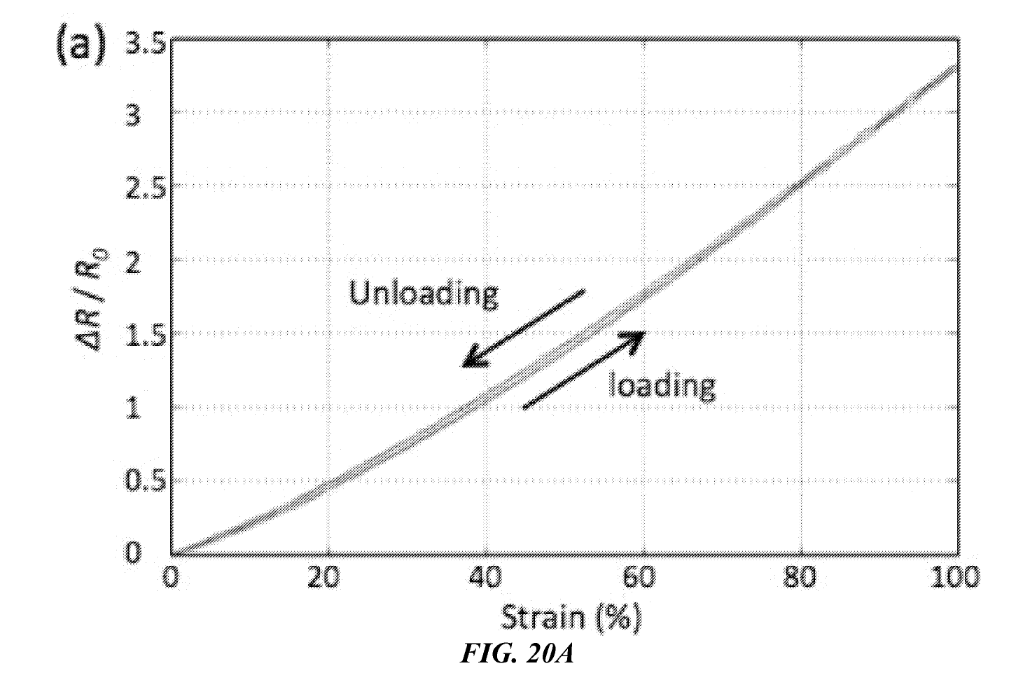

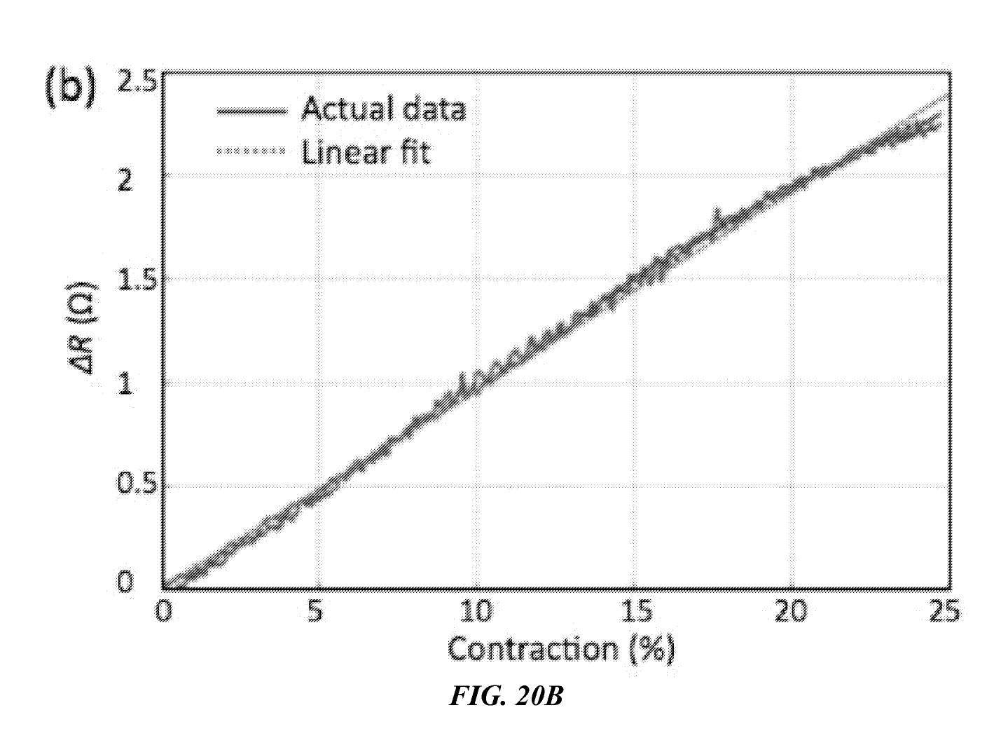

The strain sensor was calibrated by applying axial strain up to 100% multiple times using a materials tester before being integrated with an actuator, as shown in FIG. 18B. To check the hysteresis level of the sensor, the experiment included loading and unloading loops. The stretch and release rate was 0.5 mm/sec. FIG. 20A shows the calibration result. The nominal resistance at rest is 10.3Ω. Assuming the thermal effect on the strain sensor is negligible, the experimental gauge factor is 3.37 based on the calibration result. The result shows a linear and repeatable strain response and negligible hysteresis.

Once integrated with an actuator, as shown in FIG. 18C, the strain sensor signal can be used to measure the contraction of a single muscle. FIG. 20B shows the calibration result of a single sensor-actuator unit. The artificial muscle was contracted multiple times up to 25% of the original length from the relaxed state, and the resistance change of the strain sensor was measured. The result shows a linear response for measuring muscle contraction.

### Device

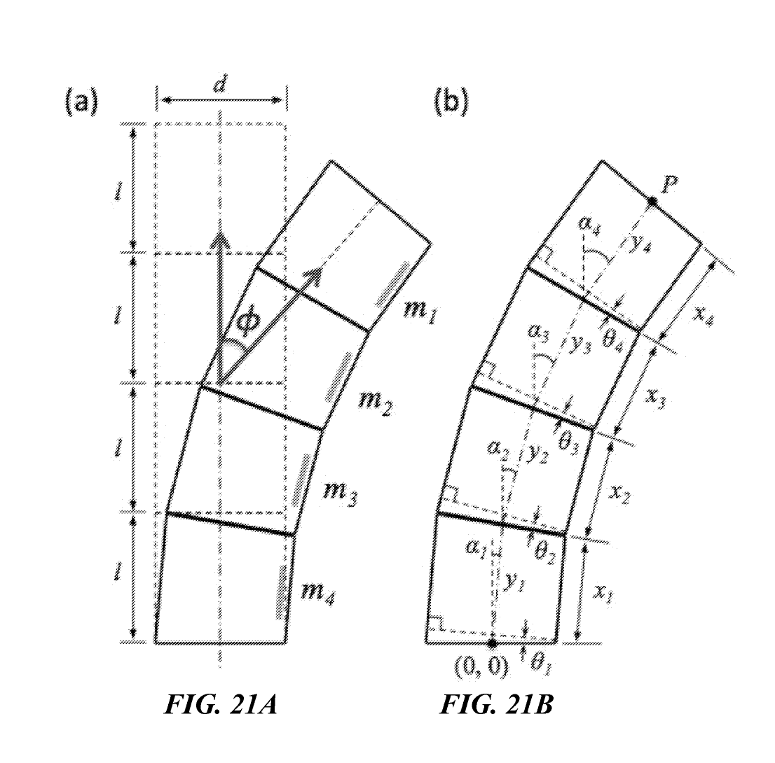

The integrated device was characterized using four pneumatic muscles, in one column, which are equipped with strain sensors. The bending angle, φ, defined in FIG. 21A, can be theoretically calculated from the diagram shown in FIG. 21B. If the length of the four contraction sides are x₁, x₂, x₃, and x₄, respectively, assuming there are no length changes on the other sides (l) and the diameter of the cylinder (d), the location of the top center point, P, can be expressed relative to the bottom center point, (0, 0) in FIG. 21, of the cylinder as following:

$$P = ( {p}_{x} , {p}_{y} ) = ( {\sum }_{i = 1}^{4} {y}_{i} \cdot sin {α}_{i} , {\sum }_{i = 1}^{4} {y}_{i} \cdot cos {α}_{i} ) \\ where \\ {y}_{i} = \frac{{x}_{i} + l}{2} , \\ {α}_{1} = {θ}_{1} , and {α}_{n} = 2 {\sum }_{i = 1}^{n - 1} {θ}_{i} + {θ}_{n} ( i \geq 2 ) . \qquad ( 5 )$$

Then, the bending angle of the entire cylinder is:

$$φ = {tan}^{- 1} ( \frac{{p}_{x}}{{p}_{y} - 2 l} ) . \qquad ( 6 )$$

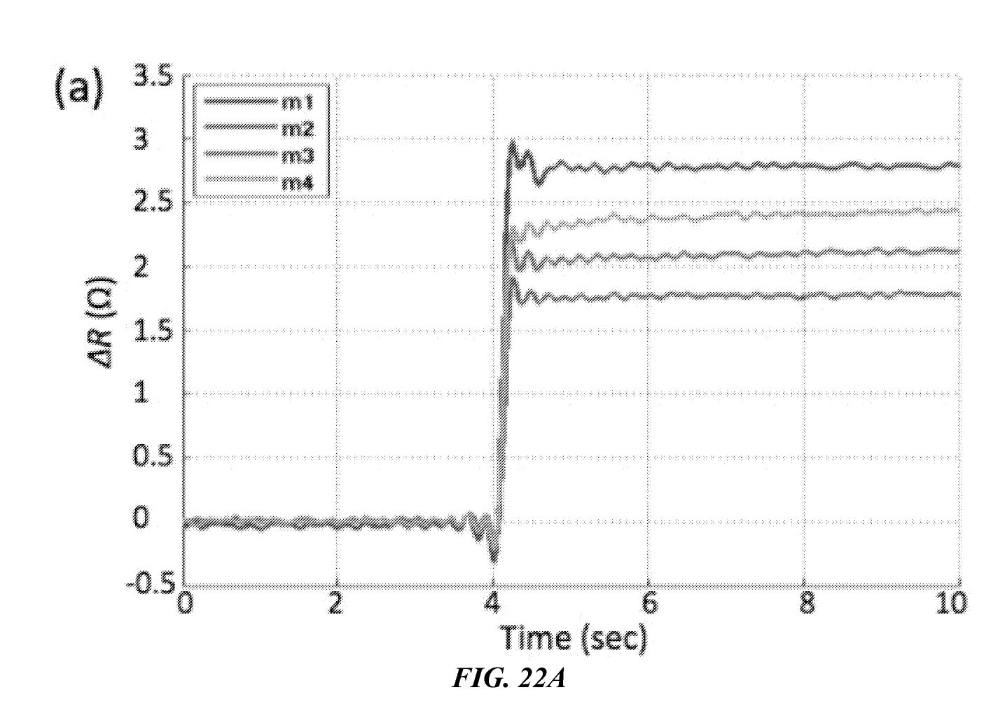

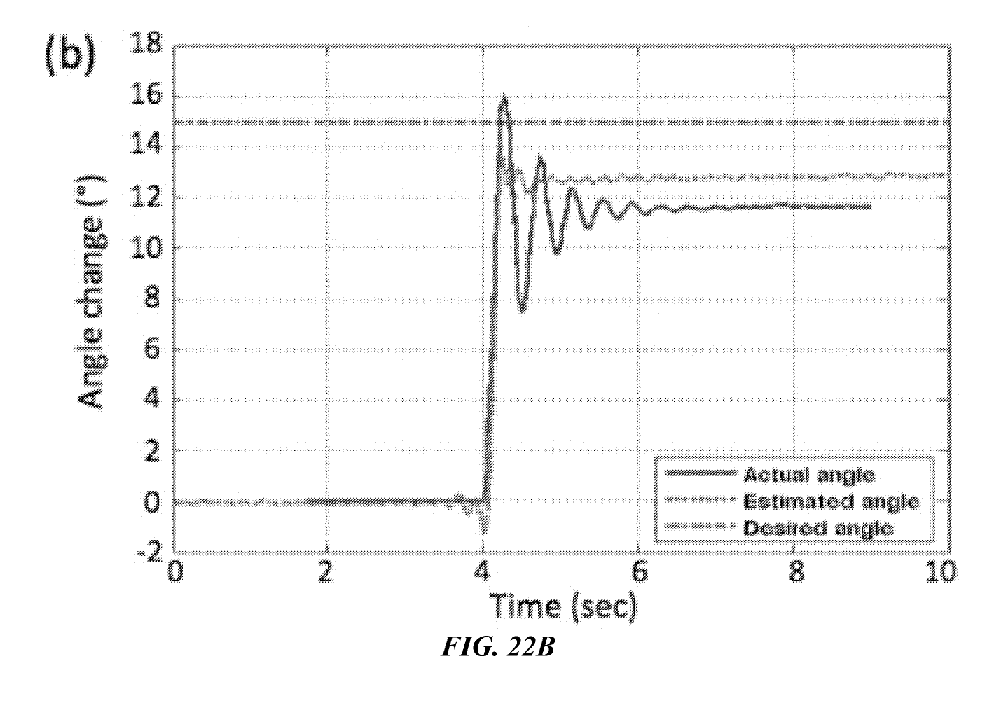

Different bending angles were achieved in the following experiments. The angles are presented in three ways in the results (FIGS. 22 to 25): desired angles that were theoretically calculated, estimated angles using the strain sensor information, and actual angles measured using a commercial video analysis software (for example, ProAnalyst, Xcitex, Cambridge, Mass. 02141, USA). The source air pressure for actuation was 500 kPa with a flow rate of 0.5 cfm during the device characterization experiments.

1) Case 1: Contract four muscles simultaneously: All four muscles are simultaneously contracted to reach the desired target angle. FIGS. 22A and 22B show the result. The target angle is 15°, the estimated angle using the strain sensors is approximately 13°, and the actual achieved angle is approximately 11.5°.

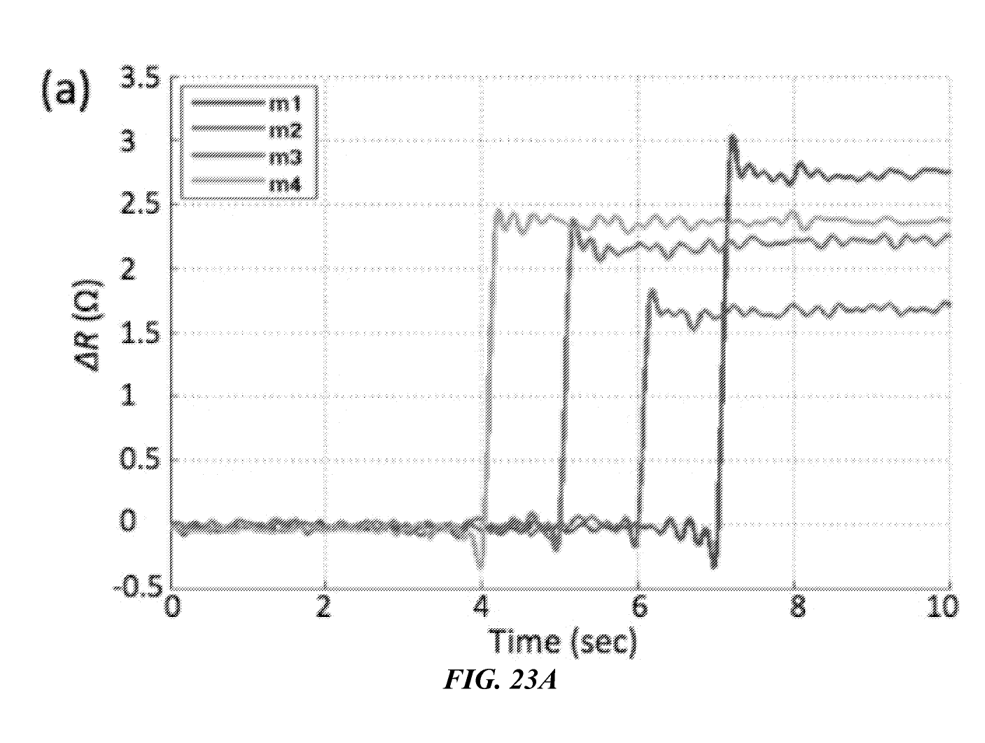

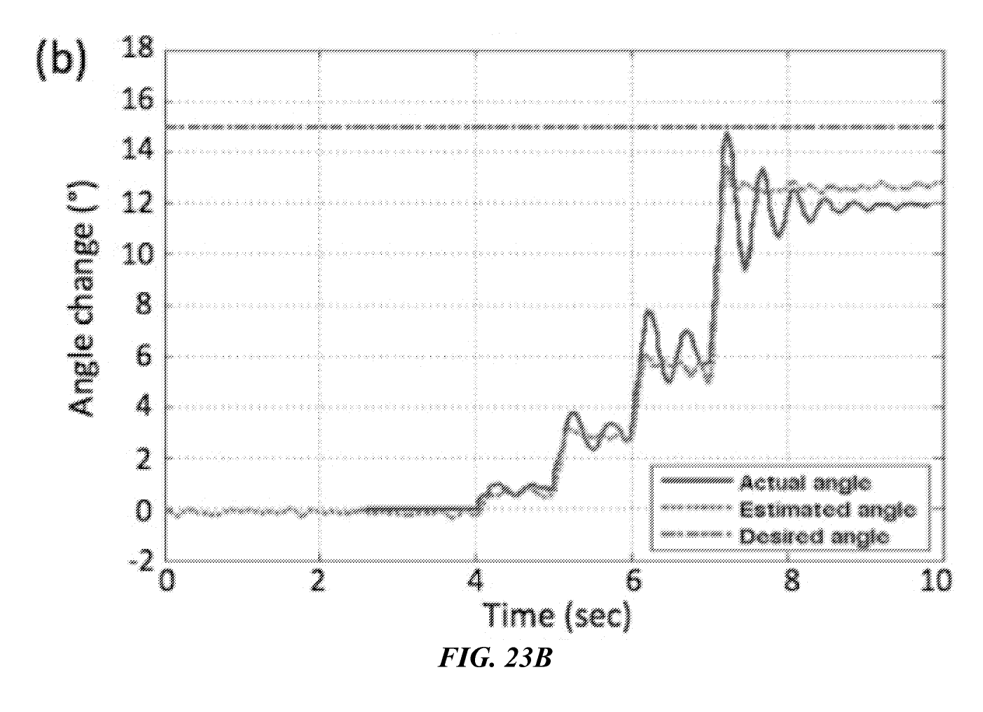

2) Case 2: Contract four muscles in sequence: In this test, the four muscles are contracted in sequence. The strain sensor data in FIG. 23A shows the sequence of the contraction. Since all four muscles are contracted eventually, the target angle is the same 15°. The estimated angle is approximately 13°, and the actual achieved angle is approximately 12°, as shown in FIG. 23B. The data show that the muscles, as expected, contracted with 1 sec interval between consecutive actuations.

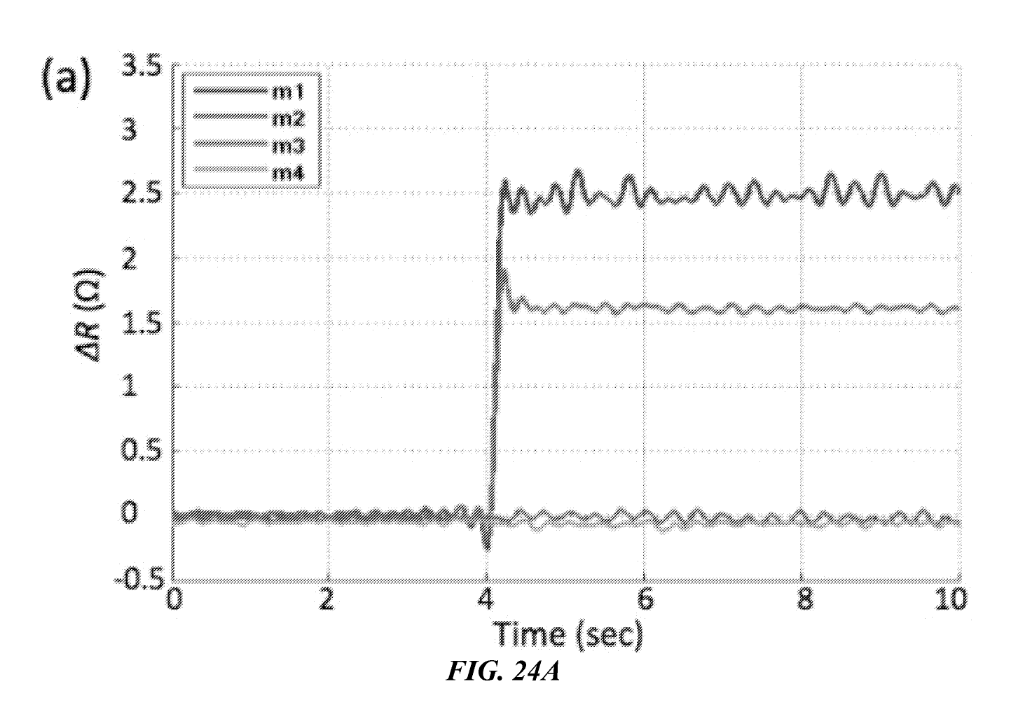

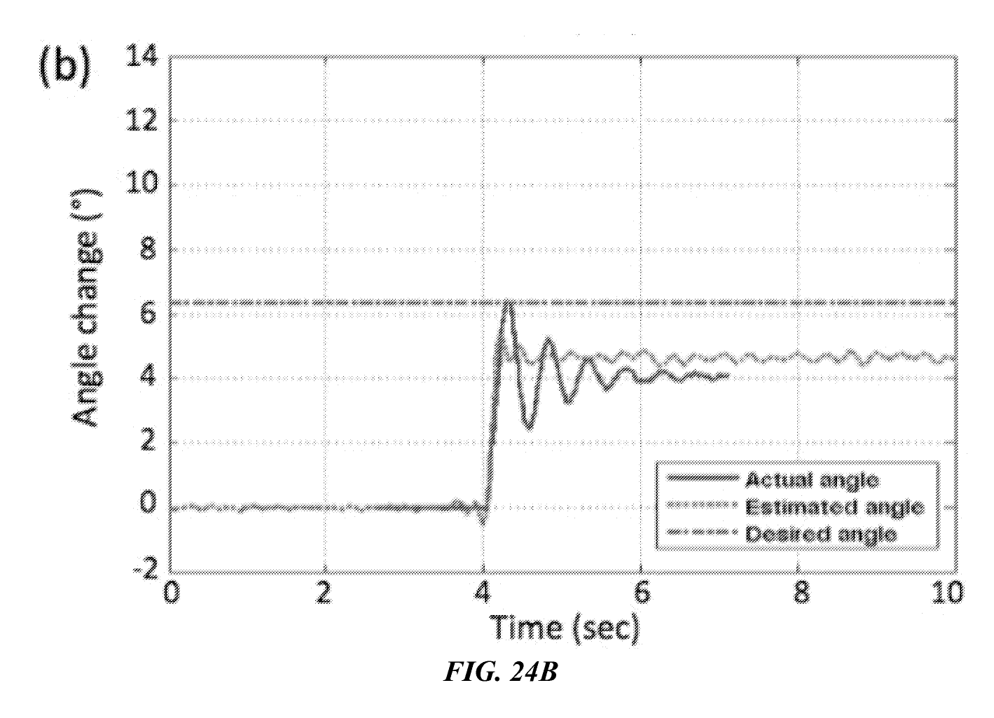

3) Case 3: Contract muscles 1 and 3 only: Only Muscles 1 and 3 are contracted. FIGS. 24A and 24B show the result. The target angle is 6.5°, the estimated angle is approximately 4.5°, and the actual achieved angle is approximately 4°.

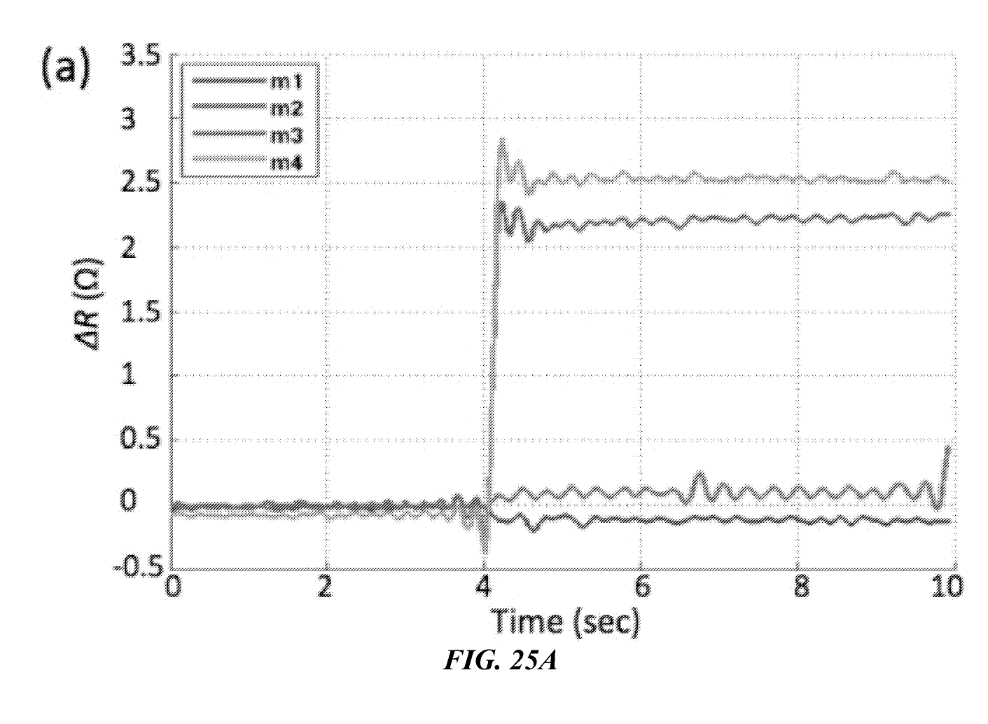

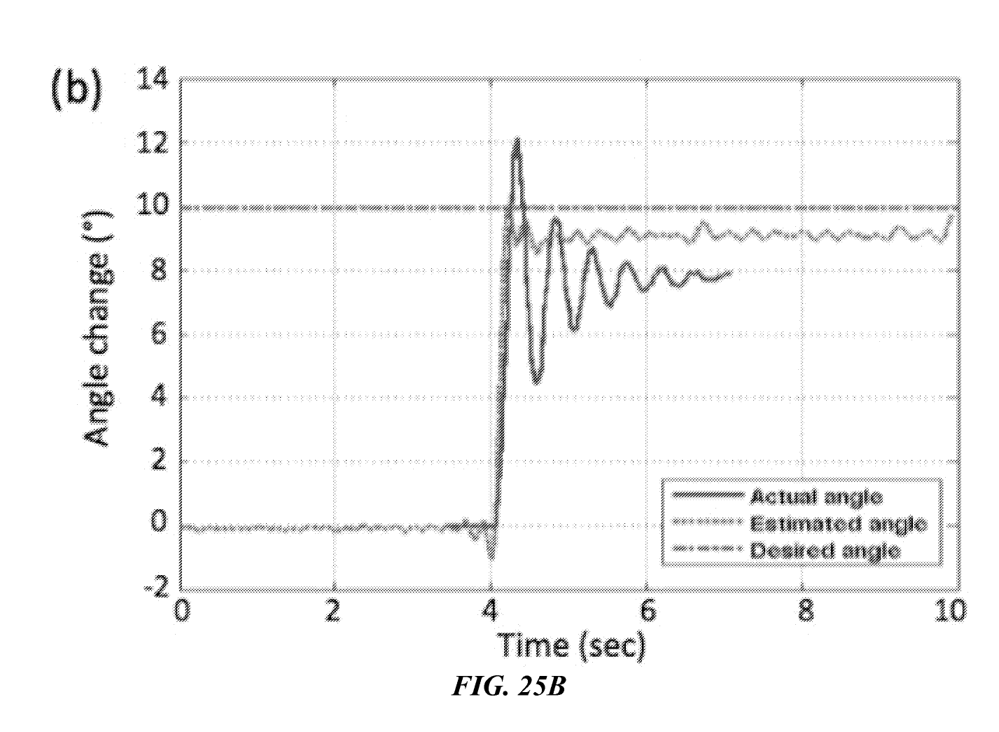

4) Case 4: Contract muscles 2 and 4 only: Muscles 2 and 4 are contracted instead of Muscles 1 and 3 in this test. FIGS. 25A and 25B show the result. The target angle is 10°, the estimated angle is approximately 9°, and the actual achieved angle is approximately 8°. Although the number of contracted muscles are the same as in Case 3, since muscles 2 and 4 are located in lower positions than muscles 1 and 3, the result shows higher achieved angle than in Case 3.

The maximum achievable bending angle was approximately 12° with contraction of four actuators in one column, and the peak torque the device can generate is approximately 6.5 Nm with a simultaneous contraction of two actuator columns. The actual achieved angles were always smaller than the estimated angles from the strain sensor signals, and the estimated angles were always smaller than the target angles calculated theoretically. There are two main reasons on these undershoot results. First, since each actuator can be hand-made, all the actuators are not exactly in the same dimensions and performance. More controlled manufacturing methods minimize this effect. Second, the robotic device 1400 does not maintain exactly the same diameter along the body length because of the hollow elastomer structure. Embedding a diagonal mesh of flexible but inextensible fibers reduces this effect while still allowing structural softness and flexibility.

The oscillations shown in all the experimental data are from the waving effect of the device. The momentary contractions of the pneumatic actuators cause the entire device to shake, since the device has its own mass and can be made of elastic material. However, once the device is wrapped around a human body, this oscillation effect can be significantly reduced.

### Discussion

The proposed orthotic has two key characteristics: softness and modularity. Being soft makes the orthotic suitable for compliance matching with natural mechanics of a person's body. Its modularity further allows the orthotic to be reconfigurable, failure tolerant, and easily tailorable to different individuals.

Although the device can be a hollow cylindrical structure made of a soft material, it still has several limitations to overcome to be fully wearable. First, the solenoid valves and relatively stiff air tubing materials reduce the wearability. Second, electric wires used in the robotic device 1400 are not stretchable. Finally, the base material, silicone rubber, is not a breathable material. In alternate embodiments, the device can be formed with soft valves and a stretchable electronics design using conductive micro-fluidic channels. Also, the device can utilize breathable materials.

Also, when an elastomer material is used for the device 1400, the resulting structure is stretchable, durable and skinlike. In addition to elastomer material, composite or hybrid materials may be used that, for example, embed fabric and elastomer together, or combine elastomer and fabric in multiple layers. One advantage of a composite or hybrid structure is that the device can allow the skin under the device to breathe and can allow moisture due to sweat to wick or evaporate.

The experiments in the “CHARACTERIZATION” section (above) show that, leveraging of modular nature of the device, the software can achieve the similar actuation goals through multiple actuation plans. This is possible because the device is constructed from networked, independently controllable, and miniaturized muscles. This highlights an important feature: the orthotic can tolerate failures within the device. That is, when a particular actuation plan fails due to temporary or permanent damages of individual muscles within the device, the controller program can be designed such that it adapts to the failures by choosing an alternative actuation plan chosen to still achieve the target length or angle changes. To realistically benefit from this property, the controller programs must be designed to monitor if target actuation goals are reached, detect problematic units preventing successful actuation, and quickly switch to an alternative plan to compensate failures of a cell. In the present invention, the design includes robust control strategies that take advantage of this property.

One important finding with the robotic device 1400 is that each individual muscle functions practically as a binary actuator due to the friction between the latex tube and the mesh sleeve. Significant axial contraction only happens when the pressure within the muscle overcomes the friction. In alternate embodiments of this device, an alternative design allows proportional actuation of each individual muscle. Actuation at finer granularities allows a bio-inspired design, in which the collective contributions of small, individual forces are added, or “recruited”, until friction is overcome, and the actuation goal is achieved.

Although the robotic device 1400 relies on the lab-bench air source for pneumatic muscle actuation, alternate embodiments can be equipped with a small compressed air canister as an air source that makes the entire system fully portable.

### CONCLUSION

A modularized programmable active sleeve was developed using pneumatic actuators, hyper-elastic strain sensors, and an elastomer sheet. The robotic device 1400 has, for example, 16 embedded pneumatic muscle cells and, for example, 4 hyper-elastic strain sensors. With different combinations of the actuations, the device is able to form various shapes in order to assist body motions. This is an initial step for building a wearable adaptive soft orthotic device. To achieve the final vision of adaptively and safely assisting human body movement, continuing research effort is necessary in developing adaptive control strategies, scalable manufacturing methods, and further miniaturization of muscle cells.

The present invention is directed to an orthotic system, comprising: a flexible material including two or more actuation modules; each actuation module including at least one controller and at least one actuator controlled by the at least one controller of the actuation module.

The orthotic system may further comprise a communication link connecting a first actuation module to a second actuation module, wherein the first actuation module can communicate with the second actuation module and cause the second actuation module to contract.

The present invention is directed to a robotic system comprising: a structural system; an actuation system; and a sensing system, wherein the actuation system and the sensing system can be embedded within the structural system.

The structural system can comprise a flexible and elastic base material.

The structural system can comprise silicone elastomer.

The structural system can comprise a silicone elastomer sheet material.

The structural system exhibits approximately the same mechanical compliance as natural skin.

The structural system can comprise a breathable material.

The actuation system can comprise an actuator that exerts tension and enables adaptive rigidity control.

The actuation system can comprise a series of pneumatically-powered actuators.

The series of pneumatically-powered actuators can comprise McKibben-type actuators.

The actuation system can comprise a muscle cell.

The muscle cell can be pneumatically actuated.

The muscle cell can comprise fibers.

The fibers can comprise fixed-length Kevlar fibers.

The fibers can be circumferentially embedded in a secondary structural system.

The secondary structural system can comprise a flexible polymer.

The flexible polymer can be provided in a tube shape.

The actuation system can have an axial shape, wherein the actuation system can be expandable, and wherein expansion of the actuation system can be constrained in an axial direction.

The actuation system can comprise an air chamber embedded between a pair of elastic layers, and a thread embedded between the pair of elastic layers.

The thread can be a Kevlar thread.

The sensing system can be coupled to a control system to enable the sensing system to detect motion of the actuation system.

The sensing system can comprise a strain sensor.

The actuation system can comprise a secondary structural system having an axial shape, wherein the strain sensor can be placed perpendicular to an axis of the secondary structural system having the axial shape.

The strain sensor can detect a change in strain of at least one layer.

The strain sensor can detect a change in strain of a layer of the structural system.

The sensing system can comprise micro-channels embedded in a layer, wherein the micro-channels can be filled with a liquid.

The layer can comprise silicone rubber.

The liquid can comprise Eutectic Gallium Indium liquid.

The micro-channels can have a width of about 250 μm and a height of about 250 μm.

The sensing system can comprise a hyper-elastic strain sensor.

The sensing system can comprise a Whitney strain gauge.

The robotic system can further comprise a control system operatively connected to the actuation system and the sensing system.

The actuation system can utilize fluid flow, and wherein the control system can comprise a valve that can control flow of a fluid into and out of the actuation system.

The valve can be a solenoid valve.

The control system can receive information from the sensing system indicative of engagement of the actuation system.

The control system can comprise a network of programmable embedded controllers.

The control system can comprise at least two controllers in communication with each other using a two-wire serial protocol, current loop, USB or Firewire.

The control system can comprise control software.

The control software can comprise a global service layer and a local service layer.

The control software can comprise a system services layer and an application program layer.

The global service layer can comprise a shape detection system or a formation algorithm.

The local service layer can comprise fundamental software components or modules that manage local resources and provide high level primitives to support algorithms at the global service layer.

The local service layer can establish timing, handles communication, issues commands to the actuation system and can receive information from the sensing system.

The system services layer can provide system primitives and can establish global time synchronization.

The application program layer can provide control program services.

The control program services can comprise data acquisition, signal processing, control logic and actuation.

The control system can control the motion and shape of the robotic system.

The control system can comprise a timing system.

The timing system can divide time into fixed rounds.

The fixed rounds can comprise fixed-order stages.

The fixed-order stages can comprise a sense stage, a fetch stage, a process stage, an actuate stage and an emit stage.

The control system can comprise a leader election and spanning tree formation algorithm.

The control system can comprise a shape detection algorithm.

The control system can comprise a shape formation algorithm.

The control system can control rigidity and contraction of the structural system by sending signals to the actuation system.

The control system can monitor and assists with human motion by receiving signals from the sensing system and sending signals to the actuation system.

The system services layer can implement fundamental components that manage local resources and provide primitives to support algorithms at the application program layer.

The system services layer can comprise one or more of the following: a clock-driven scheduler system, a messaging system, a data communication system, a system for exchanging information with the sensing system, and a system for exchanging information with the actuation system.

The control system can detect failures of the robotic system to achieve a target length or a target angle, wherein the control system adapts to the failures by choosing an alternative actuation plan chosen to still achieve the target length or angle changes.

The control system can monitor if target actuation goals can be reached, can detect problematic units preventing successful actuation, and switches to an alternative plan to compensate the failures of a cell.

The present invention is directed to a method of manufacturing an orthotic system, comprising: forming at least one controller and at least one actuator within a flexible material; operatively connecting the at least one controller to the at least one actuator.

The method can further comprise forming a communication link between a first actuation module and a second actuation module, wherein the first actuation module can communicate with the second actuation module and cause the second actuation module to contract.

The present invention is directed to a method of manufacturing a robotic system comprising: forming a structural system; embedding an actuation system within the structural system; and embedding a sensing system within the structural system.

The present invention is directed to a method of manufacturing a robotic system comprising: selectively bonding two or more layers of flexible material to form a channel permitting the flow of fluid and a pocket connected to the channel which expands and contracts as the fluid can be forced into or drawn out of the pocket; embedding a structural member within the two or more layers of flexible material; bonding a sensor to the pocket, wherein the sensor can be adapted to measure a change in a condition of the channel, the pocket, the structural member or the two or more layers of flexible material.

A first node comprising one of a plurality of channels, one of a plurality of pockets, and one of a plurality of structural members can be formed in the two or more layers of flexible material; and wherein a second node comprising another one of the plurality of channels, another one of the plurality of pockets, and another one of the plurality of structural members can be formed in the two or more layers of flexible material.

The method of manufacturing the robotic system can further comprise forming a controller operatively connected to the first node and the second node.

A first controller can be operatively connected to the first node, the second node, a third node and a fourth node to form an array of nodes that can, along with other arrays, selectively contract portions of the robotic system.

The flexible material can be conformable to a form of a human body segment.

The structural system can comprise a material that can be conformable to a form of a human body segment.

The present invention is directed to a method of manufacturing a robotic system, comprising: casting an elastomer sheet with a plurality of actuators and a plurality of sensors; connecting the plurality of actuators in series with at least one fiber to form an actuator group; connecting a plurality of actuator groups in parallel in a flat mold; connecting the plurality of sensors with at least one wire; and pouring a liquid polymer into the flat mold to form the elastomer sheet.

The method of manufacturing the robotic system can further comprise: curing the elastomer sheet into a generally cylindrical shape.

The method of manufacturing the robotic system can further comprise: curling the elastomer sheet; placing the elastomer sheet into a semicylindrical mold; forming a seam with a liquid polymer to form a generally cylindrical shape.

The method of manufacturing the robotic system can further comprise: assembling a plastic cap to an end of the cylindrical shape and fixing the fiber to the plastic cap.

The method of manufacturing the robotic system can further comprise: assembling a plastic cap to an end of the cylindrical shape and fixing the fiber to the plastic cap.

While the present invention can have been described in connection with a number of exemplary embodiments, and implementations, the present inventions can be not so limited, but rather cover various modifications, and equivalent arrangements.

### BEST MODE

Embodiments of the best modes of the invention are set forth in FIGS. 13-25 and in the description associated with the same.

### INDUSTRIAL APPLICABILITY

The present invention is applicable in the following industries: medicine, orthotics, robotics, mechanical engineering, computer science, electrical engineering, software engineering, manufacturing, commercial products, consumer products, industrial products, medical equipment and the like.

1. An orthotic system, comprising:
a flexible material including a first actuation module and a second actuation module, each actuation module being configured to change a structural characteristic of a portion of the flexible material;
each actuation module including at least one controller and at least one actuator controlled by the at least one controller of the actuation module; and
a communication link connecting the first actuation module to the second actuation module, wherein the first actuation module can communicate with the second actuation module and cause the second actuation module to change a structural characteristic of a portion of the flexible material.

2-136. (canceled)

137. The orthotic system of claim 1, further comprising a control system operatively connected to at least one of the first actuation module and the second actuation module.

138. The orthotic system of claim 137, wherein the control system comprises a network of controllers.

139. The orthotic system of claim 137, wherein the control system comprises at least two controllers in communication with each other using a two-wire serial protocol, current loop, USB or Firewire.

140. The orthotic system of claim 137, wherein the control system comprises control software executed by one or more controllers.

141. The orthotic system of claim 140, wherein the control software comprises a local service layer adapted to control at least one actuation module to produce changes in one or more structural characteristics of the flexible material by controlling at least one actuator of the at least one actuation module.

142. The orthotic system of claim 141 wherein the changes in at least one of the one or more structural characteristics of the flexible material and causes the orthotic system to move.

143. The orthotic system of claim 140, wherein the control software comprises a global service layer in communication with a local service layer to control at least one of the actuation modules to produce changes in one or more structural characteristics of the flexible material.

144. The orthotic system of claim 140, wherein the control software comprises a global service layer in communication with a local service layer to control at least two of the actuation modules to control at least two actuators to produce changes in one or more structural characteristics of the flexible material.

145. The orthotic system of claim 140 wherein the control software comprises a global service layer in communication with a local service layer to control at least two of the actuation modules to control at least two actuators to produce changes in one or more structural characteristics of the flexible material and causing a predefined motion of the orthotic system.

146. The orthotic system of claim 140, wherein the control software comprises a system services layer adapted to control at least one actuation module to produce changes in one or more structural characteristics of the flexible material by controlling at least one actuator of the at least one actuation module.

147. The orthotic system of claim 146 wherein the changes in at least one of the one or more structural characteristics of the flexible material and causes the orthotic system to move.

148. The orthotic system of claim 140, wherein the control software comprises an application program service layer in communication with a system services layer to control at least one of the actuation modules to produce changes in one or more structural characteristics of the flexible material.

149. The orthotic system of claim 140, wherein the control software comprises an application program service layer in communication with a system services layer to control at least two of the actuation modules to control at least two actuators to produce changes in one or more structural characteristics of the flexible material.

150. The orthotic system of claim 140 wherein the control software comprises an application program service layer in communication with a system services layer to control at least two of the actuation modules to control at least two actuators to produce changes in one or more structural characteristics of the flexible material and causing a predefined motion of the orthotic system.

151. The orthotic system according to claim 140 wherein at least one actuation module includes at least one sensor adapted to indicate to the control system a change in a structural characteristic of the flexible material.

152. The orthotic system according to claim 151 wherein the control system receives signals from the at least one sensor indicative of a change in a structural characteristic of the flexible material and controls at least one actuation module to arrange the flexible material in a predefined shape.

153. The orthotic system according to claim 151 wherein the control system receives signals from the at least one sensor indicative of a change in a structural characteristic of the flexible material and controls at least one actuation module to move the flexible material in a predefined motion.

154. The orthotic system according to claim 151, wherein the orthotic is worn on a person and the control system monitors and assists with human motion by receiving signals from the at least one sensor and sending signals to at least one actuation module.

155. The orthotic system according to claim 138, further comprising a plurality of actuation modules and the control system controls the actuation of each module of the orthotic system according to a predefined pattern of actuation.

156. The orthotic system according to claim 155, wherein the predefined pattern of actuation causes the flexible material to form a predefined shape.

157. The orthotic system according to claim 155, wherein the predefined pattern of actuation causes the flexible material to move according to predefined motion.

158. The orthotic system according to claim 155, wherein the predefined pattern of actuation changes over time and causes the flexible material to move.
